(19) United States
(12) Patent Application Publication
(10) Pub. No.: US 2013/0344023 A1
(43) Pub. Date: Dec. 26, 2013

(54) POLYMER-ACTIVE AGENT CONJUGATES

(71) Applicants: Michel Wathier, Madison, WI (US); Hideki Suzuki, Madison, WI (US)

(72) Inventors: Michel Wathier, Madison, WI (US); Hideki Suzuki, Madison, WI (US)

(73) Assignee: Flex-Biomedical, Madison, WI (US)

(21) Appl. No.: 13/981,235

(22) PCT Filed: Jan. 11, 2012
(86) PCT No.: PCT/US12/20959

§ 371 Date: Sep. 9, 2013

Related U.S. Application Data
(60) Provisional application No. 61/435,400, filed on Jan. 24, 2011.
(60) Provisional application No. 61/468,255, filed on Mar. 28, 2011.

(51) Int. Cl. A61K 47/48

(52) U.S. Cl. CPC A61K 47/48176
USPC 424/78.19; 525/327.2; 525/379; 525/351

(57) ABSTRACT

A polymer includes at least one monomeric unit represented by Formula I, II, or III: where X is CH₂ or O; R¹ is H, COOR², COCH₃, CONHR², OR² or SR²; L is a bond or a linker group; AA is a group derived from an active agent; each R² is independently H, a cation, an alkyl, an alkenyl, an alkynyl, COCH₃, CH₂CH₂OH, CH₂CH₂OR³, an amino acid, a small peptide, a large peptide, enzyme, protein, growth factor, cytokine, antibody, single chain antibody, antibody fragment, COCCH₃═CH₂, COCH═CH₂, CH₂CO₂H, CH₂CH₂CO₂H, CH₂CH₂SH, CH₂CH₂SR³ or (CH₂CH₂O)_{n′}R⁴; n′ is an integer from 1 to 2000; each R³ is independently trityl, 4-methyltrityl or 2 pyridyl; each R⁴ is independently H, an alkyl, an alkenyl, an alkynyl, COCCH₃═CH₂, COCH═CH₂, CH₂CHO, CH₂CH₂CHO, CO₂H, CO₂R⁵, CH₂CO₂H, CH₂CH₂CO₂H, CH₂NH₂, CH₂NHR⁵, CH₂N(R⁵)₂, CH₂CH₂NH₂, CH₂CH₂NHR, CH₂CH₂N(R⁵)₂, SH, CH₂CO₂R⁵, or CH₂CH₂CO₂R⁵; and each R⁵ is independently maleimide, a cation, an amino acid, a small peptide, a large peptide, protein, enzyme, growth factor, cytokine, antibody, single chain antibody, phosphate, sulfate, choline, or an activated ester.

### CROSS-REFERENCE TO RELATED APPLICATIONS

This application claims the benefit of U.S. Provisional Application Nos. 61/468,255, filed Mar. 28, 2011, and 61/435,400, filed Jan. 24, 2011, both of which are incorporated herein by reference, in their entirety, for any and all purposes.

### FIELD

This invention is generally related to the delivery of active agents via polymers and the use of polymers in pharmaceutical and biomedical applications.

### BACKGROUND

Osteoarthritis (OA), a non-inflammatory joint disease characterized by degeneration of joint cartilage, can affect one or more parts of the body, including hands and weight-bearing joints such as knees, hips, feet and the spine. When healthy, cartilage allows bones to glide over each other and has a shock absorbing function. In osteoarthritis, the surface layer of the cartilage breaks down and wears away, thereby allowing the surface of two bones, normally interspersed by cartilage to rub against each other causing the common OA symptoms of pain, swelling, and in more severe cases the loss of motion of the joint. In joints such as the knees, osteoarthritis is often accompanied by loss of viscosity of the synovial fluid, a thick, gel-like substance that cushions the joint and provides lubrication to reduce friction of the bones.

Osteoarthritis is mainly associated with ageing, with a prevalence of approximately 80% in individuals over 65. Despite being a condition that causes most problems to populations after retirement age, osteoarthritis is also rated the highest cause of work loss in the U.S. and Europe. In addition to age, risk factors known to be associated with osteoarthritis include obesity, traumatic injury and overuse due to sports and occupational stresses.

There is currently no cure for osteoarthritis, and available arthritis therapies are directed to symptomatic relief of pain, and at improving, or at least maintaining, joint function. Generally, pain relievers such as non-steroidal anti-inflammatory drugs (NSAIDs) or COX-2 inhibitors may be used, along with physical therapy to treat osteoarthritis.

Viscosupplementation, a procedure involving the injection of gel-like substances (generally hyaluronates or called hyaluronic acid) into a joint to supplement the viscous properties of synovial fluid, has been shown to relieve pain in many osteoarthritis patients who do not get relief from analgesic drugs. The materials injected into the joint are viscosupplements. The technique has been used in Europe and Asia for several years, but was not approved in the United States for treatment of osteoarthritis by the U.S. Food and Drug Administration until 1997. In current procedures of viscosupplementation, hyaluronate preparations are injected into joints to replace or supplement the body's natural hyaluronan, a polysaccharide component of synovial fluid. The hyaluronan preparations coat the articular cartilage surface, providing a possible prophylactic barrier for the articular cartilage. However, due to a short lifetime of hyaluronate within the joint (about a couple of days), pharmaceutical preparations of hyaluronate currently available have a limited benefit to the patient and require injection of large quantities of the preparation and/or repeated injections.

### SUMMARY

In one aspect, a polymer is provided including at least one monomeric unit represented by Formula I, II, or III:

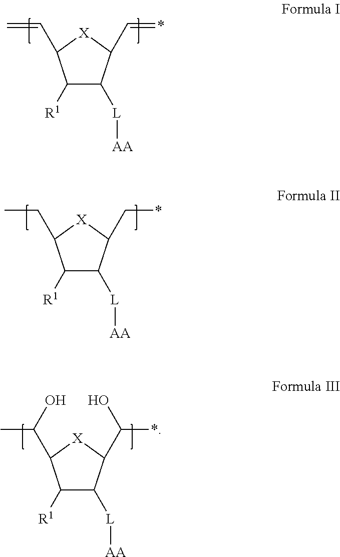

In Formulae I, II, and III, X is CH₂ or O; each R¹ is independently H, COOR², COCH₃, CONHR², OR² or SR²; each R² is independently H, a cation, an alkyl, an alkenyl, an alkynyl, COCH₃, CH₂CH₂OH, CH₂CH₂OR³, an amino acid, a small peptide, a large peptide, COCCH₃═CH₂, COCH═CH₂, CH₂CO₂H, CH₂CH₂CO₂H, CH₂CH₂SH, CH₂CH₂SR³ or (CH₂CH₂O)_{n′}R⁴.

For compounds according to Formulae I, II, or III, each R³ is independently trityl, 4-methyltrityl or 2 pyridyl; each R⁴ is independently H, an alkyl, an alkenyl, an alkynyl, COCCH₃═CH₂, COCH═CH₂, CH₂CHO, CH₂CH₂CHO, CO₂H, CO₂R⁵, CH₂CO₂H, CH₂CH₂CO₂H, CH₂NH₂, CH₂NHR⁵, CH₂N(R⁵)₂, CH₂CH₂NH₂, CH₂CH₂NHR, CH₂CH₂N(R⁵)₂, SH, CH₂CO₂R⁵, or CH₂CH₂CO₂R⁵; each R⁵ is independently maleimide, a cation, an amino acid, a small peptide, a large peptide, protein, enzyme, growth factor, cytokine, antibody, single chain antibody, phosphate, sulfate, choline, or an activated ester; L includes a bond or a linker group; AA is a group derived from an active agent; and n′ is an integer from 1 to 2000. In some embodiments, R¹ is H, C(O)OH, C(O)OR², or OR². In some embodiments, X is CH₂. In some embodiments, X is O. In some embodiments, L includes a bond. In other embodiments, L includes a linker group of formula —C(O)—R⁶—, —C(O)OR⁶—, —C(O)NH—R⁶—, —C(O)NH—R⁶—NH—, where R⁶ is an alkylene, alkylene oxide, alkylene sulfide, poly(alkylene oxide). In some embodiments, R² is H or alkyl. In some embodiments, the cation is an alkali metal, an alkaline earth metal, or an ammonium salt. In some embodiments, the cation is Li, Na, K, Cs, Ca, Mg, or Ba. In some embodiments, the cation is sodium.

In another aspect, a pharmaceutical composition including an effective amount of at least one polymer of above polymers is provided with at least one pharmaceutically acceptable carrier.

In another aspect, a method is provided including administering an effective amount of at least one of the above polymers to a subject in need thereof.

In yet another aspect, a process is provided including contacting a first polymer of Formula V, VI, or VII with a compound of Formula VIII to produce a second polymer having at least one monomeric unit of Formula IC, IIC, or IIIC; where:

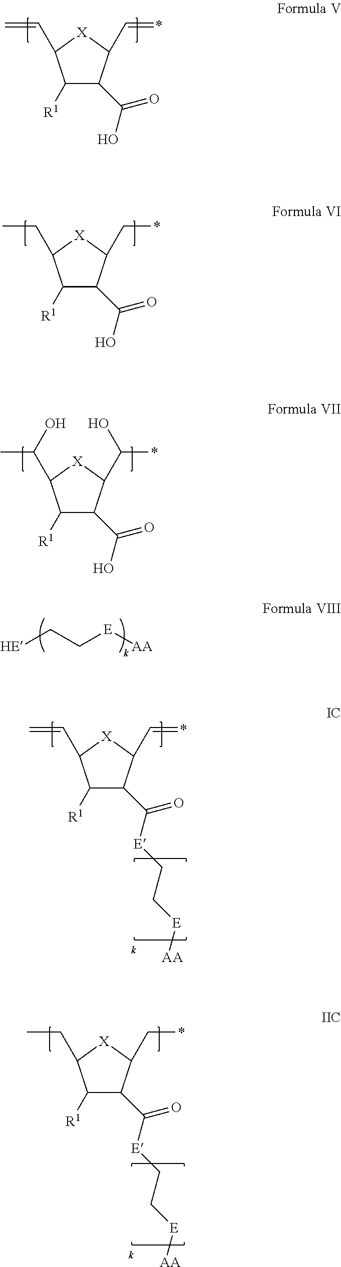

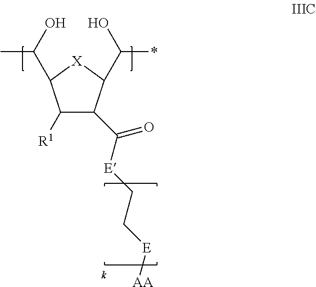

X is CH₂ or O; R¹ is H, COOR², COCH₃, CONHR², OR² or SR²; E is O, S, or R¹⁰; E′ is O, S, or NR¹⁰ or ⁺NR¹⁰HX′; X′ is an anion; k is an integer from 1 to 500; AA is a group derived from an active agent; each R² is independently H, a cation, an alkyl, an alkenyl, an alkynyl, COCH₃, CH₂CH₂OH, CH₂CH₂OR³, an amino acid, a small peptide, a large peptide, COCCH₃═CH₂, COCH═CH₂, CH₂CO₂H, CH₂CH₂CO₂H, CH₂CH₂SH, CH₂CH₂SR³ or (CH₂CH₂O)_{n′}R³, n′ is an integer from 1 to 2000; each R³ is independently trityl, 4-methyltrityl or 2 pyridyl; each R⁴ is independently H, an alkyl, an alkenyl, an alkynyl, COCCH₃═CH₂, COCH═CH₂, CH₂CHO, CH₂CH₂CHO, CO₂H, CO₂R⁵, CH₂CO₂H, CH₂CH₂CO₂H, CH₂NH₂, CH₂NHR⁵, CH₂N(R⁵)₂, CH₂CH₂NH₂, CH₂CH₂NHR, CH₂CH₂N(R⁵)₂, SH, CH₂CO₂R⁵, or CH₂CH₂CO₂R⁵; each R⁵ is independently a cation, maleimide, an amino acid, a small peptide, a large peptide, protein, enzyme, growth factor, cytokine, antibody, single chain antibody, phosphate, sulfate, choline, or an activated ester; and R¹⁰ is H or alkyl. In some embodiments, the cation is an alkali metal, an alkaline earth metal, or an ammonium salt. In some embodiments, the cation is Li, Na, K, Cs, Ca, Mg, or Ba. In some embodiments, the cation is Na. Where in Formula VIII, where the compound is a salt, an anion is present. Such salts may include any known anion. For example, in such an embodiment, the salt counter ion may be F, Cl, Br, I, trifluoromethanesulfonate, hexafluorophosphate, trifluoroacetate, tetrafluoroborate, sulfate, phosphate, carbonate, hydrogencarbonate, acetate, p-toluenesulfonate, or other pharmaceutically acceptable anions.

In another aspect, a process for synthesizing a Formula VIII compound is provided including contacting an active agent with a compound of Formula QE′(CH₂CH₂E)ₖH to form a compound of Formula QE′(CH₂CH₂E)ₖAA wherein the AA is bound to E through a —C(O)— group; and acidifying the compound of Formula QE′(CH₂CH₂E)ₖAA to form a compound of Formula VIII;

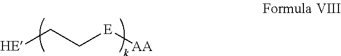

wherein the active agent comprises a carboxylic acid group; Q is a protecting group; E is O, S, or NH; E′ is O, S, NR¹⁰ or ⁺NR¹⁰HX′; X′ is an anion; and k is an integer from 1 to 500. In some embodiments, E′ is NH.

### BRIEF DESCRIPTION OF THE DRAWING

FIG. 1 is a graph of bone score in joints using a canine model of osteoarthritis, after treatment a polymer-ibupropen conjugate having a molecular weight of about 2,000,000 g/mol at a concentration of 40 mg, versus a control of commercially available Synvisc One®. The following scoring was used: 0=normal; 1=up to 10% of femur width has thickened trabeculae; 2=11-30% of femur width has thickened trabeculae; 3=31-60% of femur width has thickened trabeculae; 4=61-90% of femur width has thickened trabeculae; 5=>91% of femur width has thickened trabeculae.

### DETAILED DESCRIPTION

As used herein, the following definitions of terms shall apply unless otherwise indicated.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The terms “individual” and “subject” are used herein interchangeably. They refer to a human or another mammal (e.g., primates, dogs, cats, goats, horses, pigs, mice, rabbits, and the like). In certain preferred embodiments, the subject is human. The terms do not denote a particular age, and thus encompass adults, children, and newborn.

The term “treatment” is used herein to characterize a method or process that is aimed at (1) delaying or preventing the onset of a disease or condition; (2) slowing down or stopping the progression, aggravation, or deterioration of the symptoms of the disease or condition; (3) bringing about ameliorations of the symptoms of the disease or condition; or (4) curing the disease or condition. A treatment may be administered prior to the onset of the disease, for a prophylactic or preventive action. Alternatively or additionally, the treatment may be administered after initiation of the disease or condition, for a therapeutic action.

The term “local”, when used herein to characterize the delivery, administration or application of a polymer, or a pharmaceutical composition thereof, is meant to specify that the polymer or composition, is delivered, administered or applied directly to the site to be treated or in the vicinity of the site to be treated for a localized effect. For example, a polysaccharide mimic used as a viscosupplement will generally be injected directly to an osteoarthritic knee joint; a polysaccharide mimic used as tissue space filler will generally be injected directly to a diseased or damaged vocal cord, or to a skin area displaying lines or wrinkles. Preferably, local administration is effected without any significant absorption of components of the polysaccharide mimic into the patient's blood stream (to avoid a systemic effect).

A “pharmaceutical composition” is defined herein as comprising an effective amount of at least one active ingredient (e.g., a polymer as described herein), and at least one pharmaceutically acceptable carrier. The pharmaceutical composition can further contain one or more therapeutic drugs that are clinically used in the treatment of osteoarthritis.

As used herein, the term “pharmaceutically acceptable carrier” refers to a carrier medium which does not interfere with the effectiveness of the biological activity of the active ingredient(s) and which is not excessively toxic to the host at the concentration at which it is administered. The term includes solvents, dispersion media, coatings, antibacterial and antifungal agents, isotonic agents, absorption delaying agents, and the like. The use of such media and agents for pharmaceutically active substances is well known in the art (see for example, “Remington's Pharmaceutical Sciences”, E. W. Martin, 18.sup.th Ed., 1990, Mack Publishing Co.: Easton, Pa., which is incorporated herein by reference in its entirety).

As used herein, the term “effective amount” refers to any amount of a molecule, compound or composition that is sufficient to fulfill its intended purpose(s), i.e., to elicit a desired biological or medicinal response in a tissue or subject. Examples of intended purposes of the polymer include, but are not limited to, to provide viscosupplementation to a joint, to provide chondroprotection, to allow soft tissue augmentation, to prevent or reduce adhesion formation, to facilitate tissue manipulation, and/or to maintain, support or protect soft tissue.

As used herein, the term “soft tissue augmentation” include, but is not limited to, dermal tissue augmentation; filling of lines, folds, wrinkles, minor facial depressions, cleft lips and the like, especially in the face and neck; correction of minor deformities due to aging, disease, including in the hands and feet, fingers and toes; augmentation of the vocal cords or glottis to rehabilitate speech; dermal filling of sleep lines and expression lines; replacement of dermal and subcutaneous tissue lost due to aging; lip augmentation; filling of crow's feet and the orbital groove around the eye; breast augmentation; chin augmentation; augmentation of the cheek and/or nose; filling of indentations in the soft tissue, dermal or subcutaneous, due to, e.g., overzealous liposuction or other trauma; filling of acne or traumatic scars and rhytids; filling of nasolabial lines, nasoglobellar lines and infraoral lines.

As used herein, the term “soft tissue” includes all tissue of the body except bone. Examples of soft tissue include, but are not limited to, muscles, tendons, fibrous tissues, fat, blood vessels, nerves, and synovial tissues.

The terms “bioactive agent” and “biologically active agent” are used herein interchangeably. They refer to compounds or entities that alter, inhibit, activate or otherwise affect biological or chemical events. For example, bioactive agents may include, but are not limited to, vitamins, anti-cancer substances, antibiotics, immunosuppressants, anti-viral substances, enzyme inhibitors, opioids, hypnotics, lubricants, tranquilizers, anti-convulsants, muscle relaxants, anti-spasmodics and muscle contractants, anti-glaucoma compounds, modulators of cell-extracellular matrix interactions including cell growth inhibitors and anti-adhesion molecules, vasodilating agents, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, imaging agents. A more complete, although not exhaustive, listing of classes and specific drugs suitable for use may be found in “Pharmaceutical Substances: Synthesis, Patents, Applications” by A. Kleeman and J. Engel, Thieme Medical Publishing, 1999; and the “Merck Index: An Encyclopedia of Chemicals, Drugs, and Biologicals”, S. Budavari et al. (Eds), CRC Press, 1996, both of which are incorporated herein by reference.

The term “small molecule” refers to molecules, whether naturally-occurring or artificially created (e.g., via chemical synthesis) that have a relatively low molecular weight. Preferred small molecules are biologically active in that they produce a local or systemic effect in animals, preferably mammals, more preferably humans. Typically, small molecules have a molecular weight of less than about 1,500 Da. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered suitable for use with the present polysaccharide mimic polymers.

The terms “polysaccharide”, “carbohydrate”, and “oligosaccharide” are used herein interchangeably. They refer to a compound that comprises at least two sugar units, or derivatives thereof. Polysaccharides may be purified from natural sources such as plants or may be synthesized de novo in the laboratory. Polysaccharides isolated from natural sources may be modified chemically to change their chemical or physical properties (e.g., reduced, oxidized, phosphorylated, cross-linked). Carbohydrate polymers or oligomers may include, but are not limited to, natural sugars (e.g., glucose, fructose, galactose, mannose, arabinose, ribose, xylose, etc.) and/or modified sugars (e.g., 2′-fluororibose, 2′-deoxyribose, etc.). Polysaccharides may also be either straight or branched. They may contain both natural and/or unnatural carbohydrate residues. The linkage between the residues may be the typical ether linkage found in nature or may be a linkage only available to synthetic chemists. Examples of polysaccharides include, but are not limited to, cellulose, maltin, maltose, starch, modified starch, dextran, poly(dextrose), and fructose. Glycosaminoglycans are also considered polysaccharides. Sugar alcohol, as used herein, refers to any polyol such as sorbitol, mannitol, xylitol, galactitol, erythritol, inositol, ribitol, dulcitol, adonitol, arabitol, dithioerythritol, dithiothreitol, glycerol, isomalt, and hydrogenated starch hydrolysates.

An entity is herein said to be “associated with” another entity if they are linked by a direct or indirect, covalent or non-covalent interaction. In certain embodiments, the association is covalent. Desirable non-covalent interactions include hydrogen bonding, van der Walls interactions, hydrophobic interactions, magnetic interactions, electrostatic interactions, or combinations thereof.

In general, “substituted” refers to a group, as defined below (e.g., an alkyl or aryl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, carbonyls(oxo), carboxyls, esters, urethanes, thiols, sulfides, sulfoxides, sulfones, sulfonyls, sulfonamides, amines, isocyanates, isothiocyanates, cyanates, thiocyanates, nitro groups, nitriles (i.e., CN), and the like.

Alkyl groups include straight chain and branched alkyl groups having from 1 to 20 carbon atoms or, in some embodiments, from 1 to 12, 1 to 8, 1 to 6, or 1 to 4 carbon atoms. Alkyl groups further include cycloalkyl groups. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above. Where the term haloalkyl is used, the alkyl group is substituted with one or more halogen atoms.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include mono-, bicyclic and polycyclic ring systems, such as, for example bridged cycloalkyl groups as described below, and fused rings, such as, but not limited to, decalinyl, and the like. In some embodiments, polycyclic cycloalkyl groups have three rings. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Alkenyl groups include straight and branched chain and cycloalkyl groups as defined above, except that at least one double bond exists between two carbon atoms. Thus, alkenyl groups have from 2 to about 20 carbon atoms, and typically from 2 to 12 carbons or, in some embodiments, from 2 to 8, 2 to 6, or 2 to 4 carbon atoms. In some embodiments, alkenyl groups include cycloalkenyl groups having from 4 to 20 carbon atoms, 5 to 20 carbon atoms, 5 to 10 carbon atoms, or even 5, 6, 7, or 8 carbon atoms. Examples include, but are not limited to vinyl, allyl, CH═CH(CH₃), CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl, among others. Representative substituted alkenyl groups may be mono-substituted or substituted more than once, such as, but not limited to, mono-, di- or tri-substituted with substituents such as those listed above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8, 2 to 6, or 2 to 4 carbon atoms. Examples include, but are not limited to —C—CH, —C—C(CH₃), —C—C(CH₂CH₃), —CH₂C—CH, —CH₂C—C(CH₃), and —CH₂C—C(CH₂CH₃), among others. Representative substituted alkynyl groups may be mono-substituted or substituted more than once, such as, but not limited to, mono-, di- or tri-substituted with substituents such as those listed above.

Aryl, or arene, groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

The term “aryl”, as used herein, refers to stable mono- or polycyclic, unsaturated moieties having preferably 3-14 carbon atoms, each of which may be substituted or unsubstituted. Substituents include, but are not limited to, any of the substituents mentioned below, i.e., the substituents recited below resulting in the formation of a stable compound. The term aryl may refer to a mono- or bicyclic carbocyclic ring system having one or two aromatic rings including, but not limited to, phenyl, naphthyl, tetrahydronaphthyl, indanyl, indenyl and the like.

The term “heteroaryl”, as used herein refers to a stable heterocyclic or polyheterocyclic, unsaturated radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms. Heteroaryl moieties may be substituted or unsubstituted. Substituents include, but are not limited to, any of the substituents mentioned below, i.e., the substituents recited below resulting in the formation of a stable compound. Examples of heteroaryl nuclei include pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will also be appreciated that aryl and heteroaryl moieties, as defined herein, may be attached via an aliphatic, alicyclic, heteroaliphatic, heteroalicyclic, alkyl or heteroalkyl moiety and thus also include -aliphatic)aryl, -(heteroaliphatic)aryl, -(aliphatic)heteroaryl, -(heteroaliphatic)heteroaryl, -alkyl)aryl, -(heteroalkyl)aryl, -(heteroalkyl)aryl, and -heteroalkyl)-heteroaryl moieties. Thus, as used herein, the phrases “aryl or heteroaryl” and “aryl, heteroaryl, -(aliphatic)aryl, -(heteroaliphatic)aryl, -(aliphatic)heteroaryl, -(heteroaliphatic)heteroaryl, -(alkyl)aryl, -(heteroalkyl)aryl, -(heteroalkyl)aryl, and -(heteroalkyl)heteroaryl” are interchangeable.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Thiol” refers to the group R—S-alkyl wherein alkyl is defined herein, and R is either H or alkyl.

“Cyano” refers to the group —CN. “Carbonyl” refers to the divalent group —C(O)— which is equivalent to —C(═O)—. “Nitro” refers to the group —NO₂. “Oxo” refers to the atom (═O). “Sulfonyl” refers to the divalent group —S(O)₂—. “Thiol” refers to the group —SH. “Thiocarbonyl” refers to the divalent group —C(S)— which is equivalent to —C(═S)—. “Hydroxy” or “hydroxyl” refers to the group —OH.

The term “amine”, as used herein, refers to one, two, or three alkyl groups, as previously defined, attached to the parent molecular moiety through a nitrogen atom. It can refer to alkyl-NH₂, (alkyl)₂NH, or (alkyl)₃N groups. The term “alkylamino” refers to a group having the structure —NHR′ wherein R′ is an alkyl group, as previously defined; and the term “dialkylamino” refers to a group having the structure —NR′R″, wherein R′ and R″ are each independently alkyl groups. The term “trialkylamino” refers to a group having the structure —NR′R″R′″, wherein R′, R″, and R′″ are each independently alkyl groups. Additionally, R′, R″, and/or R′″ taken together may optionally be —CH₂)_{k′}— where k′ is an integer from 2 to 6. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “carboxylic acid”, as used herein, refers to a group of formula —CO₂H.

The terms “halo”, “halide”, and “halogen”, as used herein, refers to an atom selected from fluorine, chlorine, bromine, and iodine.

The term “methylol”, as used herein, refers to an alcohol group of structure —CH₂OH.

The term “heterocyclic”, as used herein, refers to a non-aromatic partially unsaturated or fully saturated 3- to 10-membered ring system, which includes single rings of 3 to 8 atoms in size and bi- and tri-cyclic ring systems which may include aromatic six-membered aryl or aromatic heterocyclic groups fused to a non-aromatic ring. Heterocyclic moieties may be substituted or unsubstituted. Substituents include, but are not limited to, any of the substituents mentioned below, i.e., the substituents recited below resulting in the formation of a stable compound. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized.

The term “acyl”, as used herein, refers to a group comprising a carbonyl group of the formula C═O. Examples of acyl groups include aldehydes, ketones, carboxylic acids, acyl halides, anhydrides, thioesters, amides, urea, carbamate, and carboxylic esters.

As used herein, the term “small peptide” refers to those peptides having a molecular weight from about 150 to 15,000 g/mol, while “large peptide” refers to those peptides having a molecular weigh of greater than 15,000 g/mol. As used herein, the term “protein(s)” refers to chains of 10 or more amino acids, and “enzymes” are proteins that catalyze a reaction. As used herein, “growth factor” refers to proteins or steroid hormones which are naturally occurring, genetically modified, or chemically modified substances that function as local regulators of biology. For example, growth factors are capable of stimulating (or inhibiting) cellular growth, proliferation and cellular differentiation. As used herein, the term “cytokine(s)” refers to small cell-signaling protein molecules. An antibody may be either naturally occurring, genetically modified, or chemically modified, is also known as an immunoglobulin and is large Y-shaped protein used by the immune system. A single chain antibody is a generally a smaller molecular weight antibody that lacks the constant Fc region found in a complete antibody.

In general, the present technology is directed to new biopolymers which mimic the properties of natural polysaccharides found in vivo, and which have attached to them a moiety, which when severed from the polymer, is an active agent to provide treatment to a subject. The biopolymers can be viscous liquids or gels, and are potential “bio-lubricants” that can find various applications in the biotechnology, pharmaceutical and medical fields. One such use is a viscosupplement. Thus, the polymer can provide relief to joint problems by cushioning the area between bones in a joint. Polymers may also be used to directly deliver an active agent to inflamed joint tissue. Delivery is accomplished by conjugating an active agents to the polymer using a cleavable linker. Illustrative active agents include, but are not limited to, anti-inflammatory, anti-infective, analgesic, anti-viral or antibacterial agent(s).

Thus, in one aspect, a polymer is provided, the polymer including at least one monomeric unit represented by Formula I, II, or III:

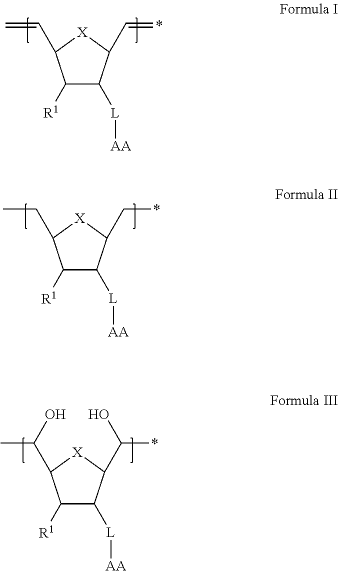

In Formulas I, II, and III, X may be a CH₂ or O group; R¹ may be H, an ester, an acid, an alkyl acid, a ketone, an amide, a alkoxy, or a thio group; L includes a bond or a linker group; and AA is a group derived from an active agent. According to some embodiments, R¹ is H, COOR², COCH₃, CONHR², OR² or SR²; where each R² is independently H, a cation, an alkyl, an alkenyl, an alkynyl, COCH₃, CH₂CH₂OH, CH₂CH₂OR³, an amino acid, a small peptide, a large peptide, COCCH₃═CH₂, COCH═CH₂, CH₂CO₂H, CH₂CH₂CO₂H, CH₂CH₂SH, CH₂CH₂SR³ or (CH₂CH₂O)_{n′}R⁴. For substituent groups having R³ and R⁴, each R³ is independently trityl, 4-methyltrityl or 2 pyridyl while each R⁴ is independently H, an alkyl, an alkenyl, an alkynyl, COCCH₃═CH₂, COCH═CH₂, CH₂CHO, CH₂CH₂CHO, CO₂H, CO₂R⁵, CH₂CO₂H, CH₂CH₂CO₂H, CH₂NH₂, CH₂NHR⁵, CH₂N(R⁵)₂, CH₂CH₂NH₂, CH₂CH₂NHR, CH₂CH₂N(R⁵)₂, SH, CH₂CO₂R⁵, or CH₂CH₂CO₂R⁵; each R⁵ is independently maleimide, an amino acid, a small peptide, a large peptide, protein, enzyme, growth factor, cytokine, antibody, single chain antibody, phosphate, sulfate, choline, or an activated ester; and n′ is an integer from 1 to 2000. Such polymers may be a viscous liquid or gel at room temperature. The monomeric, or repeat, units may be repeated one or more times in the polymer backbone. Where one or more such monomeric, or repeat, units, is included in the polymer backbone, they may be randomly ordered, or in a block arrangement. As used herein, a random co-polymer is a polymer having at least two different repeat units in no particular order and which are randomly distributed throughout the polymer backbone. A random block co-polymer is one in which there are at least two different blocks of two or more repeat units, the blocks being randomly distributed throughout the polymer backbone.

In some embodiments, R¹ is H, C(O)OH, C(O)OR², or OR², where R² is as defined above. In some other embodiments, R¹ is H. In some other embodiments, R² is H, a cation, or alkyl. In some embodiments, the cation is an alkali metal, an alkaline earth metal, or an ammonium salt. Illustrative cations include, but are not limited to, Li, Na, K, Cs, Mg, Ca, or Ba. According to one embodiment, the cation is Na. Thus, in some embodiments, R² is a cation. In other embodiments, R² is Na or K.

In some embodiments, X is O.

In some embodiments, L includes a bond. However, in other embodiments, L includes a linker group of formula —C(O)—R⁶—, —C(O)OR⁶—, —C(O)NH—R⁶—, —C(O)NH—R⁶—NH—, where R⁶ is an alkylene, alkylene oxide, alkylene sulfide, poly(alkylene oxide). In some embodiments, R⁶ is an alkylene, alkylene oxide, alkylene sulfide having from 2 to 6 carbon atoms. In some embodiments, R⁶ is a poly(alkylene oxide) which is an oligomer having from 1 to 20 ethylene oxide units. In some embodiments, R⁶ is a —(CH₂CH₂O)₃— group.

In the polymers which include at least one monomeric unit of Formulae I, II, or III. According to one embodiment, from about 0.5% to about 99% of the total monomeric units of a polymer may be selected from a Formulae I, II, or III monomeric unit. In some embodiments, the polymer which includes at least one monomeric unit of Formulae I, II, or III, can include about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the total monomeric units, a Formulae I, II, or III monomeric unit. In one embodiment, the polymer which includes at least one monomeric unit of Formulae I, II, or III, may include about 1% of the total monomeric units a Formulae I, II, or III monomeric unit. In other words, from about 1% to about 95% of the total monomeric units in the polymer include a AA group. In some embodiments, about 1% of the total monomeric units in the polymer include a AA group. In some embodiments, about 5% of the total monomeric units in the polymer include a AA group. In some embodiments, about 10% of the total monomeric units in the polymer include a AA group. In some embodiments, about 15% of the total monomeric units in the polymer include a AA group. In some embodiments, about 20% of the total monomeric units in the polymer include a AA group. In some embodiments, about 25% of the total monomeric units in the polymer include a AA group.

In Formulae I, II, and III, the AA group is an active agent moiety that when cleaved from the polymer is capable of treatment of a subject. For example, the AA group may be an active agent derived from a growth factor, a cytokine, a small molecule, an analgesic, an anesthetic, an antimicrobial agent, an antibacterial agent, an antiviral agent, an antifungal agent, an antibiotic, an anti-inflammatory agent, an antioxidant, an antiseptic agent, or a combination of any two or more such active agents. According to some embodiments, the active agent has a carboxylic acid, an ester group, an amino group, a hydroxyl group, or a thiol group carboxylic acid, an ester group, an amino group, a hydroxyl group, or a thiol group, or the active agent may be chemically modified by the addition of a carboxylic acid, an ester group, an amino group, a hydroxyl group, or a thiol group, for attaching to the polymer. Specific examples of suitable active agents are provided and discussed below.

The linking group between the polymer and the active agent moiety is a labile linking group that may be hydrolyzed in vivo, or cleaved enzymatically to release the active agent as a ketone, aldehyde, carboxylic acid, ester, or amide. Once released from the polymer, the active agent is then able to treat the subject. For example, where the active agent moiety is derived from an analgesic or anti-inflammatory, once release it may relieve pain or inflammation in and around the site where the polymer is administered.

An active agent may be selected for its ability to prevent or alleviate pain, soreness or discomfort, to provide local numbness or anesthesia, and/or to prevent or reduce acute post-operative surgical pain. Thus, suitable pain relieving agents include, but are not limited to, compounds, molecules or drugs which, when applied locally, have a temporary analgesic, anesthetic, numbing, paralyzing, relaxing or calming effect.

Suitable analgesics include, but are not limited to, non-steroidal, anti-inflammatory drugs (NSAIDs). NSAIDs have analgesic, antipyretic and anti-inflammatory activity. These compounds act peripherally and provide analgesia by interfering with the synthesis of prostaglandin, through cyclooxygenase (COX) inhibition. There are many different types of NSAIDs, including aspirin and other salicylates. Examples include, but are not limited to, ibuprofen, naproxen, sulindac, diclofenac, piroxicam, ketoprofen, diflunisal, nabumetone, etodolac, oxaprozin, and indomethacin. Aspirin is anti-inflammatory when administered in high doses, otherwise it is just a pain killer like acetaminophen. Acetaminophen has similar analgesic and antipyretic effects to the NSAIDs, but does not provide an anti-inflammatory effect. Several of the more potent NSAIDs have been developed into topical products for local administration to painful areas of the body. In some embodiments, suitable analgesics also include opioids. As used herein, the term “opioid” refers to any agonists or antagonists of opioid receptors such as the μ-, κ-, and δ-opioid receptors and different subtypes. Examples of opioids include, but are not limited to, alfentanil, allylprodine, alphaprodine, amiphenazole, anileridine, benzeneacetamine, benzoylhydrazone, benzylmorphine, benzitramide, nor-binaltorphimine, bremazocine, buprenorphine, butorphanol, clonitazene, codeine, cyclazocine, desomorphine, dextromoramide, dezocine, diampromide, dihydrocodeine, dihydrocodeine enol acetate, dihydromorphine, dimenoxadol, dimepheptanol, dimethyl-thiambutene, dioxaphetyl butyrate, dipipanone, diprenorphine, eptazocine, ethoheptazine, ethylketocyclazocine, ethylmethylthiambutene, etonitazene, etorphine, fentanyl, hydrocodone, hydromorphone, hydroxypethidine, isomethadone, ketobemidone, levallorphan, levorphanol, lofentanil, loperamide, meperidine, meptazinol, metazocaine, methadone, metopon, morphine, morphiceptin, myrophine, nalbuphine, nalmefene, nalorphine, naltrindole, naloxone, naltrexone, narceine, nicomorphine, norlevorphanol, normethadone, normorphine, norpipanone, opium, oxycodone, oxymorphone, papavereturn, papaverine, pentazocine, phenadoxone, phenazocine, phenoperidine, piminodine, piperidine, pirtramide, proheptazine, promedol, propiram, propoxyphene, remifentanil, spiradoline, sufentanil, tilidine, trifluadom, and active derivatives, prodrugs, analogs, pharmaceutically acceptable salts, or mixtures of any two or more thereof. Examples of peptide opioids include, but are not limited to, [Leu⁵]enkephalin, [Met⁵]enkephalin, Dynorphin A, Dynorphin B, α-Neoendorphin, β-Neoendorphin, βₕ-Endorphin, Deltorphin II, Morphiceptin, and active derivatives, analogs, pharmaceutically acceptable salts, or mixtures thereof.

Tricyclic antidepressants may be useful as adjuvant analgesics. These compounds are known to potentiate the analgesic effects of opioids. Tricyclic antidepressants include, but are not limited to, amitriptyline, amoxapine, clomipramine, desipramine, doxepin, imipramine, nortriptyline, protriptyline, and trimipramine.

Other suitable anesthetics include, but are not limited to, sodium-channel blockers. Examples of sodium-channel blockers include, but are not limited to, ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, etidocaine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocalne, procaine, propanocaine, proparacaine, propipocaine, propoxycaine, pseudococaine, pyrrocaine, ropivacaine, salicyl alcohol, tetracaine, tolycaine, trimecaine, zolamine, and active derivatives, prodrugs, analogs, pharmaceutically acceptable salts, or mixtures thereof.

Local anesthetics with different pharmacodynamics and pharmacokinetics may be combined in a pharmaceutical composition in order to improve the effectiveness and tolerance of the composition. For example, a composition may comprise a euctectic mixture of lidocaine and prilocalne, or a mixture of lidocaine and tetracaine. or co-administration of a glucocorticosteroid and a local anesthetic. Examples of glucocorticosteroids include, but are not limited to, dexamethazone, cortisone, hydrocortisone, prednisone, predriisolone, beclomethasone, betamethasone, flunisolide, fluocinolone, acetonide, fluocinonide, triamcinolone, and the like.

Locally acting vasoconstructive agents are also known to provide effective enhancement of local anesthesia, especially when administered through controlled release. Examples of vasoconstrictor agents include, but are not limited to, catechol amines (e.g., epinephrine, norepinephrine and dopamine); metaraminol, phenylephrine, sumatriptan and analogs, α-1 and α-2 adrenergic agonists, such as, for example, clonidine, guanfacine, guanabenz, and dopa (i.e., dihydroxyphenylalanine), methyldopa, ephedrine, amphetamine, methamphetamine, methylphenidate, ethylnorepinephrine ritalin, pemoline, and other sympathomimetic agents.

Anti-Infective Agents. Suitable anti-infective agents for use in pharmaceutical compositions are compounds, molecules or drugs which, when administered locally, have an anti-infective activity (i.e., they can decrease the risk of infection; prevent infection; or inhibit, suppress, combat or otherwise treat infection). Anti-infective agents include, but are not limited to, antiseptics, antimicrobial agents, antibiotics, antibacterial agents, antiviral agents, antifungal agents, anti-protozoan agents, and immunostimulating gents.

Suitable antiviral agents include, but are not limited to, RNA synthesis inhibitors, protein synthesis inhibitors, immunostimulating agents, and protease inhibitors. Antiviral agents may, for example, include acyclovir, amantadine hydrochloride, foscarnet sodium, ganeiclovir sodium, phenol, ribavirin, vidarabine, or zidovudine.

Examples of suitable antifungal agents include, but are not limited to, lactic acid, sorbic acid, Amphotericin B, Ciclopirox, Clotrimazole, Enilconazole, Econazole, Fluconazole, Griseofulvin, Halogropin, Introconazole, Ketoconazole, Miconazole, Naftifine, Nystatin, Oxiconazole, Sulconazole, Thiabendazole, Terbinafine, Tolnaftate, Undecylenic acid, Mafenide, Silver Sulfadiazine, and Carbol-Fushsin.

Antibiotics and other antimicrobial agents may include, but are not limited to, bacitracin; the cephalosporins (such as cefadroxil, cefazolin, cephalexin, cephalothin, cephapirin, cephradine, cefaclor, cefamandole, cefonicid, ceforanide, cefoxitin, cefuroxime, cefoperazone, cefotaxime, cefotetan, ceftazidime, ceftizoxime, ceftriaxone, and meropenem); cycloserine; fosfomycin, the penicillins (such as amdinocillin, ampicillin, amoxicillin, azlocillin, bacamipicillin, benzathine penicillin G, carbenicillin, cloxacillin, cyclacillin, dicloxacillin, methicillin, mezlocillin, nafcillin, oxacillin, penicillin G, penicillin V, piperacillin, and ticarcillin); ristocetin; vancomycin; colistin; novobiocin; the polymyxins (such as colistin, colistimathate, and polymyxin B); the aminoglycosides (such as amikacin, gentamicin, kanamycin, neomycin, netilmicin, paromomycin, spectinomycin, streptomycin, and tobramycin), the tetracyclines (such as demeclocycline, doxycycline, methacycline, minocycline, and oxytetracycline); carbapenems (such as imipenem); monobactams (such as aztreonam); chloramphenicol; clindamycin; cycloheximide; fucidin; lincomycin; puromycin; rifampicin; other streptomycins; the macrolides (such as erythromycin and oleandomycin); the fluoroquinolones; actinomycin; ethambutol; 5-fluorocytosine; griseofulvin; rifamycins; the sulfonamides (such as sulfacytine, sulfadiazine, sulfisoxazole, sulfamethoxazole, sulfamethizole, and sulfapyridine); and trimethoprim.

Other antibacterial agents include, but are not limited to, bismuth containing compounds (such as bismuth aluminate, bismuth subcitrate, bismuth subgalate, and bismuth subsalicylate); nitrofurans (such as nitrofurazone, nitrofurantoin, and furozolidone); metronidazole; timidazole; nimorazole; and benzoic acid.

Antiseptic agents may include, but are not limited to, benzalkonium chloride, chlorhexidine, benzoyl peroxide, hydrogen peroxide, hexachlorophene, phenol, resorcinol, and cetylpyridinium chloride.

The risk of infection is directly influenced by a suppressed immune system due to disease or medication. Immunostimulating agents are compounds, molecules or drugs that stimulate the immune system of a patient to respond to the presence of a foreign body, for example, by sending macrophages to the infected site(s). Suitable immunostimulating agents include a wide range of therapeutic agents, such as interleukin 1 agonists, interleukin 2 agonists, interferon agonists, RNA synthesis inhibitors, and T cell stimulating agents.

Anti-Inflammatory Agents. Suitable anti-inflammatory agents for use in pharmaceutical compositions are compounds, molecules or drugs which, when administered locally, have an anti-inflammatory activity (i.e., they can prevent or reduce the duration and/or severity of inflammation; prevent or reduce injury to cells at the injured/damaged site; prevent or reduce damage or deterioration of surrounding tissue due to inflammation; and/or provide relief from at least one of the manifestations of inflammation such as erythema, swelling, tissue ischemia, itching, fever, scarring, and the like).

Anti-inflammatory agents include, but are not limited to, NSAIDs and steroidal anti-inflammatory agents. Examples of NSAIDs can be found above. Examples of steroidal anti-inflammatory agents include, but are not limited to, aclomethasone dipropionate, flunisolide, fluticasone, budesonide, triamcinolone, triamcinoline acetonide, beclomethasone diproprionate, betamethasone valerate, betamethasone diproprionate, hydrocortisone, cortisone, dexamethason, mometasone furoate, prednisone, methylprednisolone aceponate, and prednisolone.

Anti-inflammatory agents may, alternatively or additionally, include a wide variety of compounds, molecules, and drugs exhibiting antioxidant activity. Antioxidants are agents that can prevent or reduce oxidative damage to tissue. Examples of antioxidants may include, but are not limited to, vitamin A (retinal), vitamin B (3,4-didehydroretinol), vitamin C (D-ascorbic acid, L-ascorbic acid), α-carotene, β-carotene, γ-carotene, 6-carotene, vitamin E (α-tocopherol), β-tocopherol, γ-tocopherol, 6-tocopherol, tocoquinone, tocotrienol, butylated hydroxy anisole, cysteine, and active derivatives, analogs, precursors, prodrugs, pharmaceutically acceptable salts or mixtures thereof.

In certain embodiments, the active agent is a biomolecule that is naturally present in the body and/or that is naturally secreted at an injured or damaged site (i.e., body area) and plays a role in the natural healing process. As will be apparent to those of ordinary skill in the art, variants, synthetic analogs, derivatives, and active portions of these biomolecules can, alternatively, be used in the compositions as long as they exhibit substantially the same type of property/activity as the native biomolecule. Such variants, synthetic analogs, derivatives or active portions are intended to be within the scope of the term “bioactive agents”.

Bioactive biomolecules, that have therapeutic active, include compounds that are extracted from mammalian tissues and used in a pharmaceutical composition either in crude form, or after purification. Alternatively, bioactive molecules may be prepared chemically or by conventional genetic engineering techniques, such as via expression of synthetic genes, or genes altered by site-specific mutagenesis.

Examples of suitable bioactive biomolecules include cytokines and growth factors. Cytokines and growth factors are polypeptide molecules that regulate migration, proliferation, differentiation and metabolism of mammalian cells. A diverse range of these biomolecules have been identified as potentially playing an important role in regulating healing. Examples of cytokines include, but are not limited to, interleukins (ILs) (e.g., IL-1, IL-2, IL-4 and IL-8), interferons (IFNs) (e.g., IFN-α, IFN-β, and IFN-γ), and tumor necrosis factors (e.g., TNF-α), or any variants, synthetic analogs, active portions or combinations thereof. Examples of growth factors include, but are not limited to, epidermal growth factors (EGFs), platelet-derived growth factors (PDGFs), heparin binding growth factor (HBGFs), fibroblast growth factors (FGFs), vascular endothelial growth factors (VEGFs), insulin-like growth factors (IGFs), connective tissue activating peptides (CTAPs), transforming growth factors alpha (TGF-α) and beta (TGF-13), nerve growth factor NGFs), colony stimulating factors (G-CSF and GM-CSF), and the like, or any variants, synthetic analogs, active portions or combinations thereof.

Other examples of suitable bioactive biomolecules include proteoglycans, or portions thereof. Proteoglycans are protein-carbohydrate complexes characterized by their glycosaminoglycan (GAG) component. GAGs are highly charged sulfated and carboxylated polyanionic polysaccharides. Examples of GAGs suitable for use in pharmaceutical compositions include, but are not limited to, hyaluronan, chondroitin sulfate, dermatan sulfate, heparan sulfate, and keratan sulfate.

According to yet another embodiment, suitable bioactive biomolecules include adhesion molecules. Adhesion molecules constitute a diverse family of extracellular and cell surface glycoproteins involved in cell-cell and cell-extracellular matrix adhesion, recognition, activation, and migration. Adhesion molecules are essential to the structural integrity and homeostatic functioning of most tissues, and are involved in a wide range of biological processes, including embryogenesis, inflammation, thrombogenesis, and tissue repair. Adhesion molecules include matricellular proteins (e.g., thrombospondins and tenascins), and cell surface adhesion molecules (e.g., integrins, selectins, cadherins, and immunoglobulins).

In general, the amount of bioactive agent present in the pharmaceutical composition will be the ordinary dosage required to obtain the desired result through local administration. Such dosages are either known or readily determined by the skilled practitioner in the pharmaceutical and/or medical arts.

In some embodiments, the active agent (AA) group is derived from an analgesic or an anti-inflammatory agent. For example, the AA group may be derived from an NSAID (non-steroidal anti-inflammatory drug) or a COX inhibitor. Such compounds include, but are not limited to ibuprofen, aspirin (acetylsalicylic acid), diflunisal, salsalate, naproxen, fenoprofen, ketoprofen, flurbiprofen, oxaprozin, indomethacin, sulindac, etodolac, ketorolac, diclofenac, mefenamic acid, meclofenamic acid, flufenamic acid, tolfenamic acid.

In some embodiments, the polymer is a compound of Formulae IA, IIA, or IIIA:

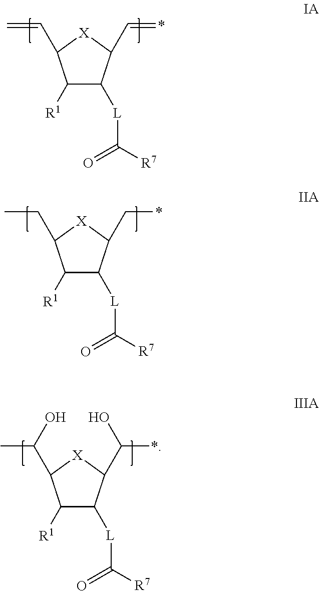

In Formulaes IA, IIA, and IIIA, L includes a linker group of Formula —C(O)—R⁶—, —C(O)OR⁶—, —C(O)NH—R⁶—, or —C(O)NH—R⁶—NH—, where R⁶ is an alkylene, alkylene oxide, alkylene sulfide, poly(alkylene oxide); and R⁷ is a alkylene group. In some embodiments, R⁷ is a group of Formula X:

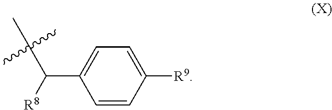

In Formula X, R⁸ may be H or alkyl; while R⁹ is alkyl. According to various embodiments, when R⁸ is alkyl, R⁸ can be methyl, ethyl, n-propyl, or iso-propyl. When R⁹ is alkyl, R⁹ may be methyl, ethyl, n-propyl, iso-propyl, n-butyl, iso-butyl, sec-butyl, tert-butyl, pentyl, neo-pentyl, iso-pentyl, or hexyl.

In some embodiments, the active agent is derived from ibuprofen and the polymer is a polymer of Formula IB, IIB, or IIIB in which the monomeric units can be present in any random order:

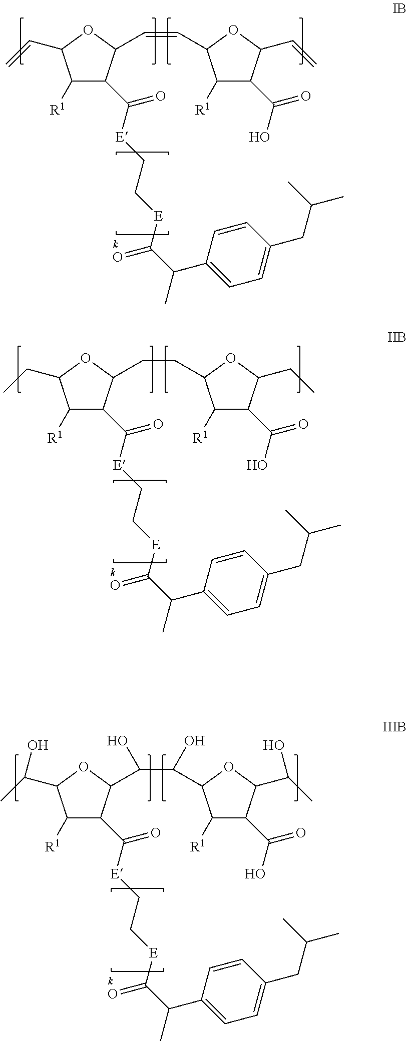

In polymers according to Formulae IB, IIB, or IIIB, R¹ may be H, C(O)OH, C(O)OR², or OR², and R² is as defined above. In Formulae IB, IIB, or IIIB, E and E′ may be S, O, NH, or NR¹⁰, where R¹ is H or alkyl, and k is an integer from 1 to 500. In some embodiments, R¹ is H, E is S and k is 1. In other embodiments, R¹ is H, E is NH and k is 1. In still other embodiments, R¹ is H, E is O and k is 1-3. In some other embodiments, E′ is NH. In other embodiments, R¹ is H, E is NH, E′ is O or NH, and k is 1. In still other embodiments, R¹ is H, E is O, E′ is O or NH, and k is 1-3. Each carboxylic acid containing monomeric, or repeat, unit, may be repeated “m” times in the polymer and each active agent containing monomeric, or repeat, unit may be repeated “j” times in the polymer. The repeat units may be randomly incorporated into the polymer, or they may be in block form, or in pairs as presented in Formulae IB, IIB, and IIIB. The formulae used herein for all polymer representations are not limiting to the exact illustrated structure of the polymer, but are simply a reflection of at least some, or all, of the repeat units in the polymer. In some embodiments, from about 1% to about 25% of the monomeric units in Formulae IB, IIB, and IIIB are monomeric units containing the ibuprofen group. In another embodiment, about 5% of the monomeric units in Formulae IB, IIB, and IIIB are monomeric units containing the ibuprofen group. In some embodiments, about 10% of the monomeric units in Formulae IB, IIB, and IIIB are monomeric units containing the ibuprofen group. In some embodiments, about 15% of the monomeric units in Formulas IB, IIB, and IIIB are monomeric units containing the ibuprofen group. In other embodiments, about 20% of the monomeric units in Formulas IB, IIB, and IIIB are monomeric units containing the ibuprofen group.

The amount of active agent in the polymer may vary. The percentage of monomeric units of the polymer which are functionalized with an active agent may range from about 0.01% to about 100%. For example, the polymers are provided that may include from about 0.5% to 100%, or from about 1% to 100% and every individual percent or range of percentages in between of monomeric polymer units functionalized with an active agent. In some embodiments, the percentage of monomeric units of the polymer which are functionalized with an active agent is about 15%, about 20%, or about 50%. In one preferred embodiment, the percentage of monomeric units of the polymer which are functionalized with an active agent is about 15%.

The polymers described above, may be viscous liquids or gels at room temperature. Such properties are dependent, at least to some extent, on the average molecular weight of the polymers. The molecular weight will be dependent to some extent on the active agent that is bound to the polymer. In some embodiments, the number average molecular weight (Mₙ) is from about 1×10⁵ g/mol to about 8×10⁶ g/mol, from about 1×10⁶ g/mol to about 7×10⁶ g/mol, from about 1×10⁶ g/mol to about 5×10⁶ g/mol, from about 2×10⁶ g/mol to about 8×10⁶ g/mol, from about 2×10⁶ g/mol to about 6×10⁶ g/mol, or from about 2×10⁶ g/mol to about 5×10⁶ g/mol. In some embodiments, Mₙ is from about 2×10⁶ g/mol to about 3×10⁶ g/mol. In some embodiments, Mₙ is from about 2.3×10⁶ g/mol to about 2.9×10⁶ g/mol. In some embodiments, Mₙ is about 2×10⁶ g/mol. In some embodiments, Mₙ is about 2.6×10⁶ g/mol.

A pharmaceutical composition includes an effective amount of at least one of the above polymers, and at least one pharmaceutically acceptable carrier or excipient. Pharmaceutical formulations can be varied depending upon the route of administration and desired dosage. Such formulations may influence the physical state, stability, rate of in vivo release, and rate of in vivo clearance of the administered compounds. Formulation will preferably produce liquid or semi-liquid (e.g., gel) pharmaceutical compositions.

Pharmaceutical compositions may be formulated in dosage unit form for ease of administration and uniformity of dosage. The expression “unit dosage form”, as used herein, refers to a physically discrete unit of polymer for the patient to be treated. Each unit contains a predetermined quantity of active material calculated to produce the desired effect. It will be understood, however, that the total dosage of the composition will be decided by the attending physician within the scope of sound medical judgment.

Formulation of pharmaceutical compositions will mainly depend on the form of administration chosen. In certain embodiments, injectable formulations (e.g., solutions, dispersions, suspensions, emulsions) will be preferred, for example, for administration to a joint (e.g., knee), an intervertebral disc, the urinary system, or the vocal cord. Injectable formulations can also be used for certain reconstruction or cosmetic procedures. Other procedures may alternatively use gels, lotions, creams, ointments, plasters, bandages, sheets, foams, films, sponges, dressings, or bio-adsorbable patches that can be applied to the area in need of treatment.

Physiologically acceptable carriers, vehicles, and/or excipients for use with pharmaceutical compositions can be routinely selected for a particular use by those skilled in the art. These include, but are not limited to, solvents, buffering agents, inert diluents or fillers, suspending agents, dispersing or wetting agents, preservatives, stabilizers, chelating agents, emulsifying agents, anti-foaming agents, ointment bases, penetration enhancers, humectants, emollients, and skin protecting agents. In some embodiments, the pharmaceutically acceptable carrier is an aqueous solution.

Examples of solvents include, but are not limited to, water, Ringer's solution, U.S.P. isotonic sodium chloride solution, alcohols, vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, and diethylamine. Suitable suspending agents include, for example, naturally-occurring gums (e.g., acacia, arabic, xanthan, and tragacanth gum), celluloses (e.g., carboxymethyl-, hydroxyethyl-, hydroxypropyl-, and hydroxypropylmethylcellulose), alginates and chitosans. Examples of dispersing or wetting agents are naturally-occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate).

Preservatives may be added to a pharmaceutical composition to prevent microbial contamination that can affect the stability of the formulation and cause infection in the patient. Suitable examples of preservatives include parabens (such as methyl-, ethyl-, propyl-, p-hydroxy-benzoate, butyl-, isobutyl- and isopropyl-paraben), potassium sorbate, sorbic acid, benzoic acid, methyl benzoate, phenoxyethanol, bronopol, bronidox, MDM hydantoin, iodopropylnyl butylcarbamate, benzalconium chloride, cetrimide, and benzylalcohol. Examples of chelating agents include sodium EDTA and citric acid.

Examples of emulsifying agents are naturally-occurring gums, naturally-occurring phosphatides (e.g., soybean lecithin, sorbitan mono-oleate derivatives), sorbitan esters, monoglycerides, fatty alcohols, and fatty acid esters (e.g., triglycerides of fatty acids). Anti-foaming agents usually facilitate manufacture, they dissipate foam by destabilizing the air-liquid interface and allow liquid to drain away from air pockets. Examples of anti-foaming agents include simethicone, dimethicone, ethanol, and ether.

Examples of gel bases or viscosity-increasing agents are liquid paraffin, polyethylene, fatty oils, colloidal silica or aluminum, glycerol, propylene glycol, carboxyvinyl polymers, magnesium-aluminum silicates, hydrophilic polymers (such as, for example, starch or cellulose derivatives), water-swellable hydrocolloids, carragenans, hyaluronates, and alginates. Ointment bases suitable for use in the pharmaceutical compositions may be hydrophobic or hydrophilic; and specific examples include paraffin, lanolin, liquid polyalkylsiloxanes, cetanol, cetyl palmitate, vegetable oils, sorbitan esters of fatty acids, polyethylene glycols, and condensation products between sorbitan esters of fatty acids, ethylene oxide (e.g., polyoxyethylene sorbitan monooleate), and polysorbates.

Examples of humectants are ethanol, isopropanol glycerin, propylene glycol, sorbitol, lactic acid, and urea. Suitable emollients include cholesterol and glycerol. Examples of skin protectants include vitamin E, allatoin, glycerin, zinc oxide, vitamins, and sunscreen agents.

In certain embodiments, pharmaceutical compositions may, alternatively or additionally, include other types of excipients including, thickening agents, bioadhesive polymers, and permeation enhancing agents.

Thickening agents are generally used to increase viscosity and improve bioadhesive properties of pharmaceutical compositions. Examples of thickening agents include, but are not limited to, celluloses, polyethylene glycol, polyethylene oxide, naturally occurring gums, gelatin, karaya, pectin, alginic acid, and povidone. In certain embodiments, a thickening agent is selected for its thioxotropic properties (i.e., has a viscosity that is decreased by shaking or stirring). The presence of such as an agent in a pharmaceutical composition allows the viscosity of the composition to be reduced at the time of administration to facilitate its application, e.g., to a skin area to be repaired, and to increase after application so that the composition remains at the site of administration.

Permeation enhancing agents are vehicles containing specific agents that affect the delivery of active components through the skin. Permeation enhancing agents are generally divided into two classes: solvents and surface active compounds (amphiphilic molecules). Examples of solvent permeation enhancing agents include alcohols (e.g., ethyl alcohol, isopropyl alcohol), dimethyl formamide, dimethyl sulfoxide, 1-dodecylazocyloheptan-2-one, N-decyl-methylsulfoxide, lactic acid, N,N-diethyl-m-toluamide, N-methylpyrrolidone, nonane, oleic acid, petrolatum, polyethylene glycol, propylene glycol, salicylic acid, urea, terpenes, and trichloroethanol. The surfactant permeation enhancing agent in the pharmaceutical compositions may be nonionic, amphoteric, cationic, anionic, or zwitterionic. Suitable nonioinic surfactants include poly(oxyethylene)-poly(oxypropylene) block copolymers, commercially known as poloxamers; ethoxylated hydrogenated castor oils; polysorbates, such as Tween 20 or Tween80. Amphoteric surfactants include quaternized imidazole derivatives; cationic surfactants include cetypyridinium chloride, “soap” (fatty acid), alkylsulfonic acid salts (the main component of synthetic detergent, such as linear alkyl benzene sulfonate (LAS)), fatty alcohol sulfate (the main component of shampoo or old neutral detergents); and zwitterionic surfactants include the betaines and sulfobetaines.

Injectable formulations can be sterilized, for example, by filtration through a bacterial-retaining filter, by incorporating sterilizing agents in the form of sterile solid compositions which can be dissolved or dispersed in sterile water or other sterile injectable medium prior to use, or by irradiation sterilization (e.g., gamma and e-beam).

Methods are provided which generally include administration of an effective amount of a polymer as described above, or a pharmaceutical composition thereof, to an individual in need thereof.

The polymers can be used as viscosupplements. As already mentioned above, viscosupplementation is a procedure involving injection of gel-like substances (generally hyaluronates, HAs) into a joint to supplement the viscous properties of synovial fluid. HA injections have been found to relieve pain in many osteoarthritis patients, with injections of HAs having a higher molecular weights (i.e., higher viscosity) showing better efficacy than injections of HA's having lower molecular weights (i.e., lower viscosity). However, due to the short lifetime of hyaluronates within the joint (about a couple of days), hyaluronate preparations currently available provide only limited long-term benefit to the patient. Current therapeutic regimens using hyaluronates overcome this disadvantage by injecting large quantities of a hyaluronate preparation into joints and/or increasing the frequency of administration of hyaluronate preparations.

The polymers may also find applications as viscoelastics useful in surgery. Viscoelastic agents used in surgery may perform a number of different functions, including, without limitation, maintenance and support of soft tissue, tissue manipulation, lubrication, tissue protection, and adhesion prevention. As will be appreciated by one skilled in the art, the rheological properties of the polymers will necessarily affect their ability to perform these functions, and, as a result, their suitability for certain surgical procedures.

Viscoelastics are, for example, used in opththalmic surgery, such as cataract surgery. Cataracts, which are opacities of the natural ocular lens, can strike people in their 40s and 50s, but they occur most commonly in those over age 60—with a rapid increase in prevalence after that. More than 50% of all Americans 65 and older have cataracts, increasing to 70% among those over 75. In order to improve eyesight, the cataractous lens is surgically removed from the eye and an artificial intraocular lens is inserted in its place. Viscoelastics were introduced in the early 1980s in response to the observation that, during cataract surgery, the underside of the cornea was often damaged due to contact with instruments, devices, fluid bubbles, and intraocular lenses. Because the cells in this region cannot regrow, there was a need to protect them. Thus, during these surgical procedures, viscoelastic materials are typically injected into the anterior chamber of the eye to prevent collapse of the anterior chamber and to protect the delicate eye tissues from damage resulting from physical manipulation. Viscoelastics also gently inflate spaces inside the eye, making it easier to maneuver various tools inside the eye.

Other examples of ocular surgical procedures that employ viscoelastics include trabeculectomy (i.e., glaucoma filtration surgery), and vitrectomy (i.e., replacement of the vitrous, a normally clear, gel-like substance that fills the center of the eye), which may be performed to clear blood and debris from the eye, to remove scar tissue, or to alleviate traction on the retina.

The polymers may also find application as tissue space fillers in any of a wide variety of soft tissue augmentation procedures, including, but not limited to, reconstruction or cosmetic enhancement, treatment for stress urinary incontinence, and treatment of vocal cord problems (e.g., paralysis, atrophy or paresis). Tissue space fillers are used to correct deformities or to reconstruct areas that are missing or defective due to surgical intervention, trauma, disease, aging, or congenital condition. Examples of reconstruction or cosmetic enhancement procedures include, but are not limited to, dermal tissue augmentation; filling of lines, folds, wrinkles, minor facial depressions, cleft lips and the like, especially in the face and neck; correction of minor deformities due to aging or disease, including in the hands and feet, fingers and toes; dermal filling of sleep lines and expression lines; replacement of dermal and subcutaneous tissue lost due to aging; lip augmentation; filling of crow's feet and the orbital groove around the eye; breast augmentation; chin augmentation; augmentation of the cheek and/or nose; filling of indentations in the soft tissue, dermal or subcutaneous, due to, e.g., overzealous liposuction or other trauma; filling of acne or traumatic scars and rhytids; filling of nasolabial lines, nasoglabellar lines and infraoral lines.

Urinary incontinence is an underserved market: there are approximately 40 million people in the U.S. that suffer from urinary incontinence, yet there are only about 250,000 procedures performed each year. Collagen bulking agents are generally used to treat urinary incontinence. They are injected into tissue surrounding the urethra to tighten the urethral sphincter and stop urine from leaking. However, these agents require several injections across multiple appointments. They also have a poor cure rate of approximately 27% to 36%. If the procedure is successful, the success is only temporary as the collagen reabsorbs into the surrounding tissue. A carbon-bead based product (Durasphere™, Advanced UroScience, Inc., Saint Paul, Minn.) entered the market in 1999 with the promise of permanence (due to less degradation of the material) but clinical data have not supported those claims and the product appears to have similar performance to collagen. Q-Med AB (Uppsala, Sweden) recently introduced Zuidex™, an HA gel which is reinforced by the addition of dextranomer, that promises immediate effects and ease of administration. New biomaterials, such as the above polymers, could impact the market if they require less material, fewer injections and had better longevity.

In vocal cord disorders such as paralysis, atrophy and paresis, one or both vocal cords are weakened and lack the ability to close and thus vibrate properly, resulting in a soft, breathy or weak voice. The affected cord may also allow food and liquids into the trachea or lungs causing difficulty with swallowing and coughing. Vocal cord paralysis may be caused by chest and neck surgery, brain injury, neck injury, lung or thyroid cancer, certain neurologic conditions, or a viral infection. In older people, vocal cord atrophy is a common problem affecting voice production. Standard treatments of vocal cord disorders include voice therapy and surgery. In surgery, doctors attempt to add bulk to the injured vocal cord by injecting a substance (e.g., fat or collagen) into the cord. This moves the injured cord closer to the non-injured cord, allowing for better contact and improved speech and swallowing. Other substances are being studied for vocal cord augmentation including silicone paste, Teflon paste, calcium hydroxylapatite, and hyaluronic acid.

The polymers may also be used as anti-adhesives. Anti-adhesives are devices that keep tissues from abnormally joining together following surgery. These abnormal unions, called adhesions, may form between an incision in the abdominal wall and the small bowel after abdominal surgery, leading to chronic pain or even bowel obstruction. Adhesions also occur following gynecological surgery, resulting in fibrous scarring that may involve the uterus, bladder, bowel or ovaries and fallopian tubes, and that can, in the worst case, lead to infertility. A wide variety of approaches, including use of steroids, non-steroidal anti-inflammatory drugs and minimally invasive surgical techniques, have been used in an attempt to prevent adhesions. However, biodegradable barriers appear to be the most promising tools available for keeping adjacent organs separate following surgery (P. B. Arnold et al., Fertil. Steril., 2000, 73: 157-161). Examples of such barriers include, but are not limited to, anti-adhesive membranes that may be laid on localized areas of the peritoneum, such as Interceed Absorbable Adhesion Barrier (Johnson & Johnson Patient Care Inc., New Brunswick, N.J.); Preclude Surgical Membrane (E.L. Gore Co., Flagstaff, Ariz.) and Seprafilm Surgical Membrane (Genzyme, Cambridge, Mass.); and viscous gels, such as Hyskon (Pharmacia, Piscataway, N.J.); Sepracoat (Genzyme) and Intergel (Lifecore Biomedical, Inc., Chaska, Minn.).

In another aspect, a method of treatment is provided where any of the above described polymers, or a pharmaceutical composition thereof, will generally be administered in such amounts and for such a time as is necessary or sufficient to achieve at least one desired result. As will be appreciated by one skilled in the art, the desired result may vary depending on the condition to be treated (e.g., osteoarthritis, cataract, dermal or subcutaneous tissue loss, urinary incontinence, or vocal cord disorder) and the purpose for which the polymer (e.g., viscosupplementation, tissue augmentation, adhesion prevention, or soft tissue maintenance, support or protection) is used. Thus, for example, in certain embodiments, a polymer of may be administered to the knee joint of a patient suffering from osteoarthritis in such amounts and for such a time that it provides pain relief, prevents or reduces swelling, prevents or reduces loss of motion of the joint and/or or improves motion of the joint. In other embodiments, a polymer may be administered to the skin of a patient undergoing a cosmetic procedure in such amounts and for such a time that lines, folds, wrinkles or minor facial depressions are filled.

Treatment protocols using the described polymers or compositions thereof may include administration of a single dose or a plurality of doses over a period of time. Administration may be once or multiple times daily, weekly (or at some other multiple day interval) or on an intermittent schedule. The exact amount of a polymer, or a pharmaceutical composition thereof, to be administered will vary from subject to subject and will depend on several factors (see below).

The polymers, or pharmaceutical compositions thereof, may be administered using any route of administration effective for achieving the desired effect. Administration will generally be local rather than systemic. Methods of local administration include, but are not limited to, dermal, intradermal, intramuscular, intraperitoneal, subcutaneous, ocular, and intra-articular routes.

Depending on the route of administration, effective doses may be calculated according to the body weight, body surface area, or organ size of the subject to be treated. Optimization of the appropriate dosages can readily be made by one skilled in the art in light of pharmacokinetic data observed in human clinical trials. Alternatively or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

According to one aspect, a method includes administering an effective amount of one or more of a polymer of Formula IA, IIA, or IIIA to a subject in need thereof. In some embodiments, the subject is a mammal. In other embodiments, the subject is a human. In some embodiments of the method, the administering includes performing local administration of an effective amount of the polymer to a tissue of the subject. In some embodiments, the tissue is a soft tissue. In some embodiments, the step of administering includes performing a single injection, while in other embodiments, the step of administering includes performing at least two injections, while in yet other embodiments, the step of administering includes performing multiple injections. In some embodiments, where two or more injections are performed, they are performed at least 6 months apart.

In some embodiments of the injection into tissue, the tissue is a diseased or injured synovial joint, and the polymer is used as a viscosupplement.

The polymer may be prepared as a pharmaceutical composition prior to injection. The composition may include the polymer and a pharmaceutically acceptable carrier. For example, the polymer may be dissolved or suspended in an aqueous vehicle. In some embodiments, the polymer is dissolved or suspended in an aqueous vehicle from about 0.1 to about 99 wt %. In other embodiments, the polymer is dissolved or suspended in an aqueous vehicle from about 0.1 wt % to about 75 wt %, from about 0.1 wt % to about 50 wt %, from about 0.1 wt % to about 25 wt %, or from about 0.1 wt % to about 15 wt %. In some embodiments of the methods of administration, the polymer is prepared as an aqueous solution from about 0.1 wt % to about 10 wt %, prior to administering.

In another aspect, a process is provided for preparation of the polymers containing the active agent moiety. Such process include contacting a first polymer including a monomeric unit of Formula V, VI, or VII with a compound of Formula VIII to produce a second polymer having at least one monomeric group of Formula IC, IIC, or IIIC. In the process, the monomeric groups referred to in the previous sentence are:

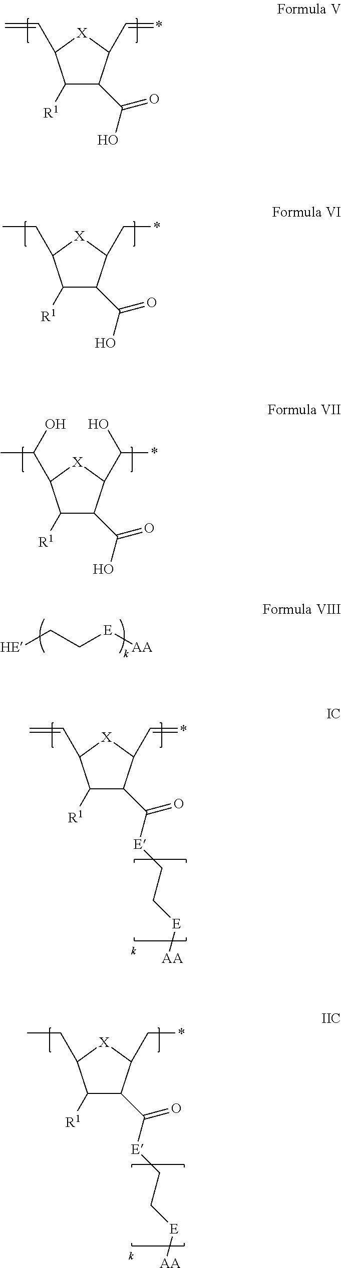

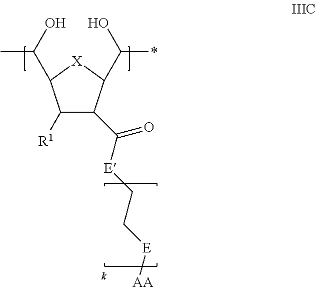

In these compounds, X is CH₂ or O; E is O, S, or NR¹⁰; E′ is O, S, NR¹⁰, or NR¹⁰HX′, where X′ is an anion and R¹⁰ is H or alkyl; k is an integer from 1 to 500; AA is a group derived from an active agent; R¹ is H, COOR², COCH₃, CONHR², OR² or SR²; each R² is independently H, a cation, an alkyl, an alkenyl, an alkynyl, COCH₃, CH₂CH₂OH, CH₂CH₂OR³, an amino acid, a small peptide, a large peptide, protein, enzyme, growth factor, cytokine, antibody, single chain antibody, antibody fragment, COCCH₃═CH₂, COCH═CH₂, CH₂CO₂H, CH₂CH₂CO₂H, CH₂CH₂SH, CH₂CH₂SR³ or (CH₂CH₂O)_{n′}R³; each R³ is independently trityl, 4-methyltrityl or 2 pyridyl; each R⁴ is independently H, an alkyl, an alkenyl, an alkynyl, COCCH₃═CH₂, COCH═CH₂, CH₂CHO, CH₂CH₂CHO, CO₂H, CO₂R⁵, CH₂CO₂H, CH₂CH₂CO₂H, CH₂NH₂, CH₂NHR⁵, CH₂N(R⁵)₂, CH₂CH₂NH₂, CH₂CH₂NHR, CH₂CH₂N(R⁵)₂, SH, CH₂CO₂R⁵, or CH₂CH₂CO₂R⁵; and each R⁵ is independently maleimide, an amino acid, a small peptide, a large peptide, protein, enzyme, growth factor, cytokine, antibody, single chain antibody, phosphate, sulfate, choline, or an activated ester; and n′ is an integer from 1 to 2000. Where in Formula VIII, E′ is —NHR¹⁰⁺X′ the compound is an ammonium salt, and the counter ion X′ is any known anion, and R¹⁰ is H or alkyl. For example, in such an embodiment, the salt counter ion may be F, Cl, Br, I, trifluoromethanesulfonate, hexafluorophosphate, trifluoroacetate, tetrafluoroborate, sulfate, phosphate, carbonate, hydrogencarbonate, acetate, p-toluenesulfonate, or other anions. As will be understood by those of ordinary skill in the art, Z is derived from E′ in Formula VIII.

In some embodiments, the compound of Formula VIII is a polyethylene glycol, where k is from 1 to 1000. In some embodiments k is from 1 to 500, from 1 to 100, from 1 to 25, or from 1 to 10. In another embodiment, k is from 1 to about 20. In yet another embodiment, k is 2, 3, 4, 5, 6, 7, 8, 9, or 10. According to one embodiment, E is O and k is 2, 3, 4, 5, 6, 7, 8, 9, or 10. In other embodiments, E′ is NH.

In some embodiments of the process, the first polymer is a compound of Formula V and the second polymer is a compound of Formula IC. In other embodiments, R¹ is H. In yet other embodiments, AA includes a carbonyl group bound to E.

In some embodiments of the process, the second polymer is of Formula ID, IID, or IIID:

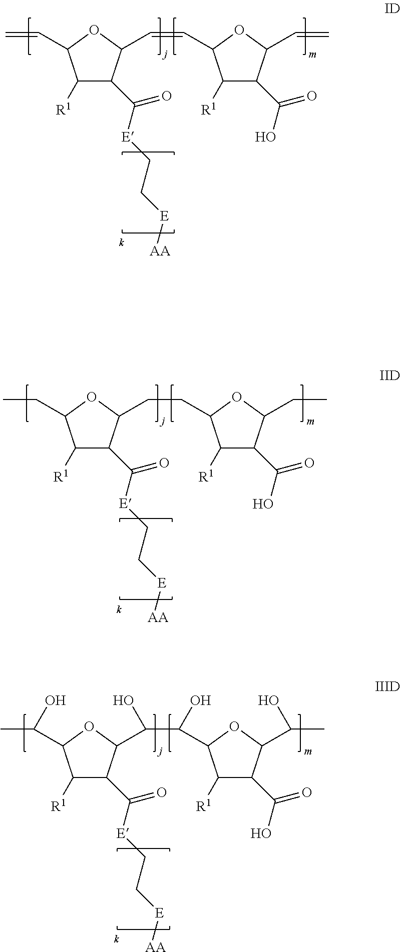

where j is from 100 to 200,000 and m is from 100 to 200,000.

In some embodiments of the process, AA is a group of formula:

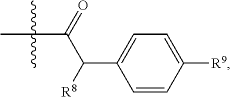

where R⁸ is H or alkyl; and R⁹ is alkyl.

In another aspect, a process is provided for preparation of the compound of Formula VIII. The process includes contacting an active agent with a compound of Formula QE′(CH₂CH₂E)ₖH to form a compound of Formula QE′(CH₂CH₂E)ₖAA wherein the AA is bound to E through a —C(O)— group; and acidifying the compound of Formula QE′(CH₂CH₂E)ₖAA to form a compound of Formula VIII. According to such embodiments, the active agent includes a carboxylic acid group; Q is a protecting group; E is O, S, or NH; E′ is O, S, or NH; and k is an integer from 1 to 500. Suitable protecting groups include, but are not limited to, those such as tert-butyloxycarbonyl (BOC), benzyloxycarbonyl, carbobenzyloxy (Cbz), p-methoxybenzyl carbonyl (Moz), 9-fluorenylmethyloxycarbonyl (FMOC), acetyl (Ac), benzoyl (Bz), benzyl (Bn), p-methoxybenzyl (PMB), benzyl 3,4-dimethoxybenzyl (DMPM), p-methoxyphenyl (PMP), and tosyl (Ts). In some embodiments, the compound of Formula VIII is a compound of Formula VIIIA:

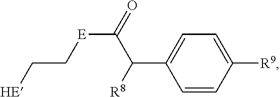

where R⁸ is H or alkyl; and R⁹ is alkyl. In some embodiments, R¹ is H, E is S and k is 1. In other embodiments, R¹ is H, E is NH and k is 1. In yet other embodiments, R¹ is H, E is O and k is 1-3. In some embodiments, E′ is O, S, or NR¹⁰. In other embodiments, E′ is NH₂ or ⁺NH₂X′.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology, thus generally described, will be understood more readily by reference to the following examples, which are provided by way of illustration and are not intended to be limiting.

### EXAMPLES

### Example 1

Synthesis of Viscosupplement carrying Amide-pro-ibuprofen. A polymer with bound ibuprofen through an amide linkage was prepared according to Scheme 1.

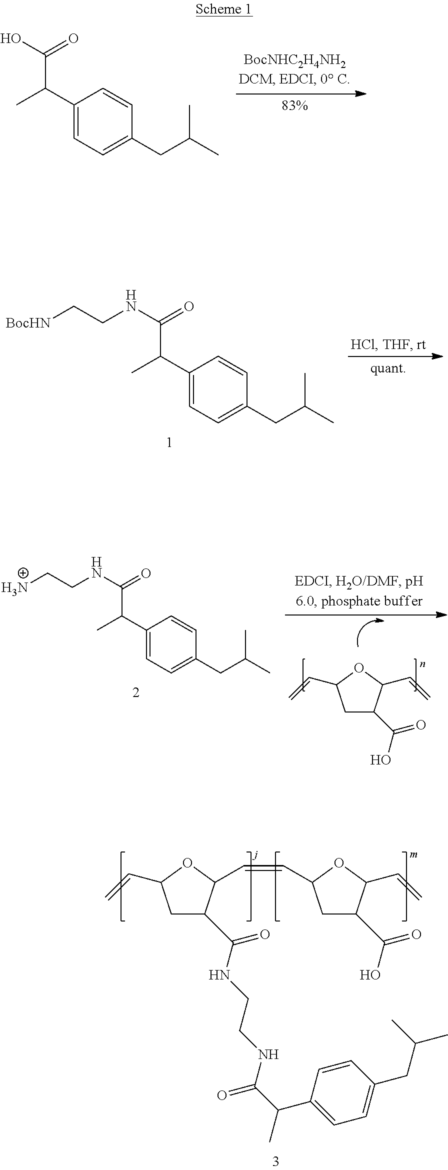

Synthesis of tert-butyl 2-(2-(4-isobutylphenyl)propanamido)ethylcarbamate (1). Ibuprofen (1 g, 4.85 mmol), tert-butyl 2-aminoethylcarbamate (1 mL, 1.008 g, 6.3 mmol), and catalytic amount of 4-dimethylamino pyridine (DMAP) were dissolved in dichloromethane (DCM; 15 mL) at 0° C. To the solution was added dicyclohexyl carbodiimide (DCC); 985 mg, 4.85 mmol), and the reaction was stirred overnight, with warming to room temperature (rt). The reaction mixture was subsequently filtered, and washed with NaHCO₃ solution. The organic layer was dried over Na₂SO₄, filtered, and the volatiles removed in vacuo. The residue was the purified by silica gel chromatography (EtOAc). The title compound I was isolated as a white powder (90% yield). ¹H NMR (300 MHz, CDCl₃) δ=0.89 (d, 6H), 1.41 (s, 9H), 1.50 (d, 3H), 1.84 (septet, 1H), 2.44 (d, 2H), 3.19 (m, 2H), 3.22 (dt, 2H), 3.85 (q, 1H), 4.95 (br s, 1H), 6.02 (br s, 1H), 7.13 (dd, 4H). ¹³C NMR (100 MHz, CDCl₃) δ=18.9, 22.1, 28.3, 31.4, 37.8, 39.6, 45.7, 45.9, 78.7, 127.5, 129.6, 137.8, 140.6, 155.7, 171.0. HRMS: m/z calcd for C₂₀H₃₁NO₄ [M+Na]⁺: 372.2151. found: 372.2156.

2-(2-(4-isobutylphenyl)propanamido)ethanaminium chloride (2). Compound 1 (1.4 g, 4.02 mmol) was dissolved in dry tetrahydrofuran (THF; 10 mL) and dry HCl (3 mL, 2M in dioxane) was added to the solution at rt. The reaction was stirred overnight, and the precipitate was collected by filtration and washed with cold ether (3×10 mL), affording 2 as a white powder in quantitative yield (1.15 g, 4.02 mmol). ¹H NMR (300 MHz, D₂O) δ=0.85 (d, 6H), 1.15 (septet, 3H), 1.65 (d, 1H), 2.99 (m, 2H), 3.2-4.4 (m, 2H), 3.60 (m, 1H), 6.75 (dd, 4H). ¹³C NMR (75 MHz, D₂O) δ=18.1, 22.3, 26.1, 29.9, 42.4, 44.9, 47.5, 128.1, 129.2, 137.2, 139.8, 175.9. HRMS: m/z calcd for C₁₅H₂₅N₂O [M]⁺: 249.1921. found: 249.1919.

Synthesis of Viscosupplement carrying amide-pro-Ibuprofen (3). Polyacid (100 mg, MW=2 MDa) was dissolved in phosphate buffer (10 mL, pH 6, 0.1M). Dimethylformamide (DMF) was added (2.5 mL), and the solution was cooled to 0° C. To the solution was added 2 (50 mg, 0.143 mmol) dissolved in H₂O (0.5 mL), followed by 1-ethyl-3-(3′-dimethylaminopropyl)carbodiimide hydrochloride (EDCI); 60 mg, 0.17 mmol) dissolved in H₂O (0.5 mL). The reaction was stirred overnight allowing it to warm to rt. The solution was precipitated into ice-cold 1M HCl (150 mL) and filtered. The precipitate was then re-dissolved in phosphate buffer (pH 7.4), and dialyzed for 96 h at 4° C. (membrane MW cut-off 10 kDa) in UltraPur water. The water was lyophilized off, affording the modified polymer as a white foam.

### Example 2

Synthesis of Viscosupplement carrying thioester-pro-Ibuprofen. A polymer with bound ibuprofen through a thioester linkage was prepared according to Scheme 2.

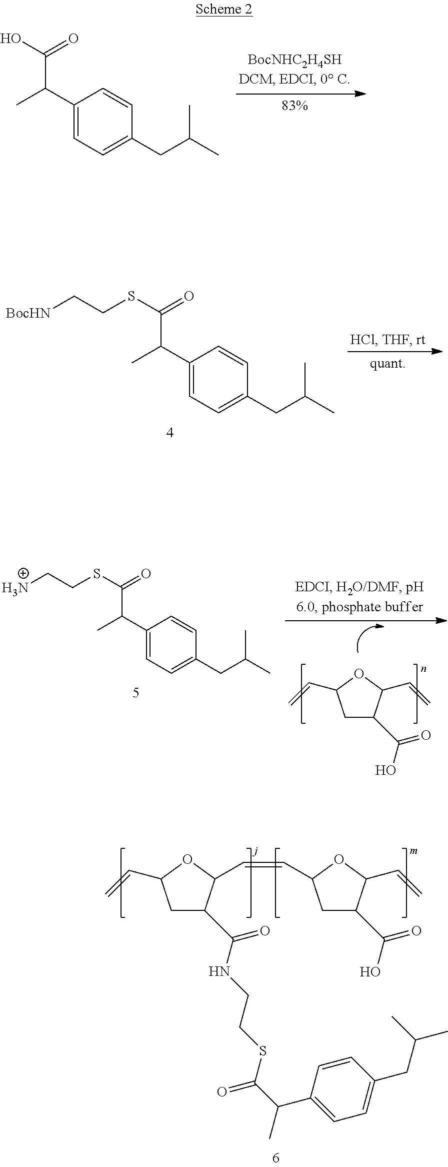

S-2-(tert-Butoxycarbonylamino)ethyl 2-(4-isobutylphenyl)propanethioate (4). Ibuprofen (1 g, 4.85 mmol), tert-butyl 2-mercaptoethylcarbamate (0.82 mL, 852 mg, 4.85 mmol), and catalytic amount of DMAP were dissolved in DCM (15 mL) at 0° C. To the solution was added DCC (985 mg, 4.85 mmol), and reaction was stirred overnight, warming to rt. The reaction was subsequently filtered, and the volatiles evaporated. The residue was purified by silica gel chromatography (hexanes:EtOAc -5:1). The product 4 was obtained as a clear oil (57% yield, 1.0 g, 2.76 mmol). ¹H NMR (300 MHz, CDCl₃) δ=0.89 (d, 6H), 1.41 (s, 9H), 1.50 (d, 3H), 1.84 (septet, 1H), 2.44 (d, 2H), 2.94 (t, 2H), 3.22 (dt, 2H), 3.85 (q, 1H), 4.88 (br s, 1H), 7.13 (dd, 4H). ¹³C NMR (75 MHz, CDCl₃) δ=18.6, 22.6, 28.5, 29.4, 30.4, 40.4, 45.2, 54.1, 79.6, 127.8, 129.6, 137.1, 141.2, 155.9, 201.4. HRMS: m/z calcd for C₂₀H₃₁NSO₃ [M+Na]⁺: 388.1917. found: 388.1911.

2-[2-(4-Isobutylphenyl)propanoylthio]ethanaminium chloride (5). Compound 4 (1.0 g, 2.76 mmol) was dissolved in dry THF (10 mL) and dry HCl (3 mL, 2M in dioxane) was added to the solution at rt. The reaction was stirred overnight, filtered, and the filtrand washed with cold ether (3×10 mL), affording 5 as a white powder in quantitative yield (833 g, 2.76 mmol). ¹H NMR (300 MHz, D₂O) δ=0.53 (d, J⁼6.3 Hz, 6H), 1.14 (d, 3H), 1.46 (septet, 1H), 2.04 (d, 2H), 2.77-2.88 (m, 1H), 2.93-3.09 (m, 3H), 3.70 (q, 1H), 6.75 (dd, 4H). ¹³C NMR (75 MHz, D₂O) δ=18.1, 22.3, 26.1, 29.9, 39.1, 44.9, 53.5, 128.0, 129.3, 137.2, 140.4, 202.9. HRMS: m/z calcd for C₁₅H₂₃NOS [M]⁺: 266.1573. found: 266.1579.

Synthesis of Viscosupplement carrying thioester-pro-Ibuprofen (6). Polyacid (100 mg, MW=2 MDa) was dissolved in phosphate buffer (10 mL, pH 6, 0.1M). DMF was added (2.5 mL), and the solution was cooled to 0° C. To the solution was added 5 (38 mg, 0.143 mmol) dissolved in H₂O (0.5 mL), followed by EDCI (30 mg, 0.17 mmol) dissolved in H₂O (0.5 mL). The reaction was stirred overnight allowing it to warm to rt. The solution was directly dialyzed for 72 h at 4° C. (membrane MW cut-off 300 kDa) in phosphate buffer (pH 6. 0.1M), and then for 48 h at 4° C. in water. The water was lyophilized off, affording the modified polymer as a white foam.

### Example 3

Synthesis of Synthesis of Viscosupplement carrying ester-pro-Ibuprofen. A polymer with bound ibuprofen through a ester linkage was prepared according to Scheme 3.

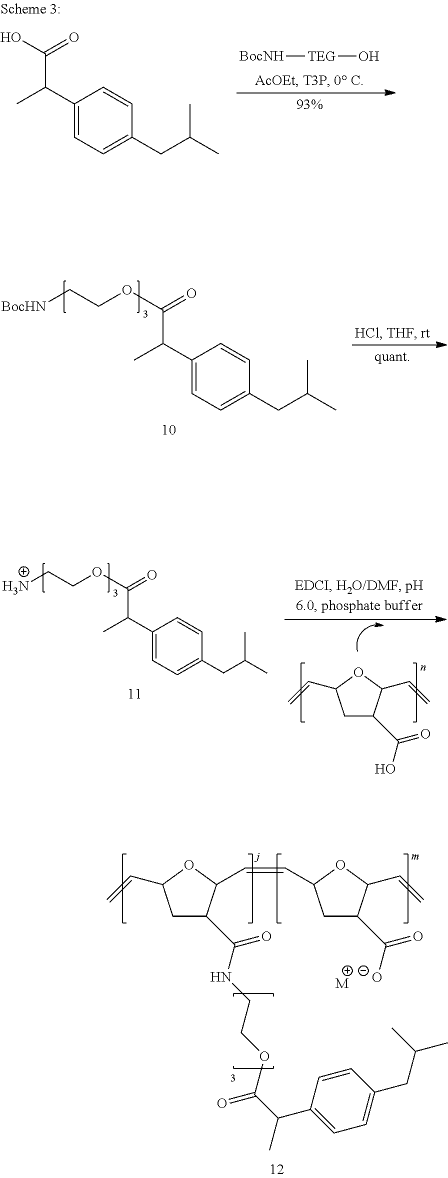

2-(tert-Butoxycarbonylamino)ethyl 2-(4-isobutylphenyl)propanoate (7). Ibuprofen (1 g, 4.85 mmol), tert-butyl 2-hydroxyethylcarbamate (1 mL, 1.02 g, 6.3 mmol), and catalytic amount of DMAP were dissolved in DCM (15 mL) at 0° C. To the solution was added DCC (985 mg, 4.85 mmol), and reaction was stirred overnight, warming to rt. The reaction was subsequently filtered, and washed with NaHCO₃ solution. Organic layer was dried over Na₂SO₄, filtered, and the volatiles evaporated. The residue was purified by silica gel chromatography (hexanes:EtOAc -7:1, 4:1). The product 7 was a clear oil (83% yield, 1.4 g, 4.02 mmol). ¹H NMR (400 MHz, CDCl₃) δ=0.89 (d, 6H), 1.43 (s, 9H), 1.49 (d, 3H), 1.84 (septet, 1H), 2.44 (d, 2H), 3.30 (br s, 2H), 3.70 (q, 1H), 4.08-4.14 (m, 2H), 4.58 (br s, 1H), 7.15 (dd, 4H). ¹³C NMR (100 MHz, CDCl₃) δ=18.5, 22.6, 28.5, 30.4, 39.8, 45.1, 45.2, 63.9, 79.7, 127.3, 129.6, 137.9, 140.8, 155.9, 174.8. HRMS: m/z calcd for C₂₀H₃₁NO₄ [M+Na]⁺: 372.2145. found: 372.2151.

2-[2-(4-Isobutylphenyl)propanoyloxy]ethanaminium chloride (8). Compound 7 (1.4 g, 4.02 mmol) was dissolved in dry THF (10 mL) and dry HCl (3 mL, 2M in dioxane) was added to the solution at rt. The reaction was stirred overnight, filtered, and the filtrand washed with cold ether (3×10 mL), affording 8 as a white powder in quantitative yield (1.15 g, 4.02 mmol). ¹H NMR (300 MHz, D₂O) δ=0.65 (d, 6H), 1.27 (d, 3H), 1.61 (septet, 1H), 2.24 (d, 2H), 3.04-3.19 (m, 2H), 3.73 (q, 1H), 4.01-4.26 (m, 2H), 7.04 (dd, 4H). ¹³C NMR (75 MHz, D₂O) δ=17.8, 21.8, 29.8, 38.4, 44.4, 44.7, 61.5, 127.4, 129.8, 137.6, 141.5, 176.7. HRMS: m/z calcd for C₁₅H₂₃NO₂ [M+H]⁺: 250.1802. found: 250.1833.

Synthesis of Viscosupplement carrying ester-pro-Ibuprofen (9). Polyacid (100 mg, MW=2 MDa) was dissolved in phosphate buffer (10 mL, pH 6, 0.1M). DMF was added (2.5 mL), and the solution was cooled to 0° C. To the solution was added 8 (41 mg, 0.143 mmol) dissolved in H₂O (0.5 mL), followed by EDCI (30 mg, 0.17 mmol) dissolved in H₂O (0.5 mL). The reaction was stirred overnight allowing it to warm to rt. The solution was precipitated into ice-cold 1M HCl (150 mL) and filtered. The filtrand was redissolved in phosphate buffer (pH 7.4), and dialyzed for 96 h at 4° C. (membrane MW cut-off 300 kDa) in UltraPur water. The water was lyophilized off, affording the modified polymer as a white foam.

### Example 4

Synthesis of Viscosupplement carrying TEG-ester-pro-Ibuprofen. A polymer with bound ibuprofen through a glycol-ester linkage was prepared according to Scheme 4.

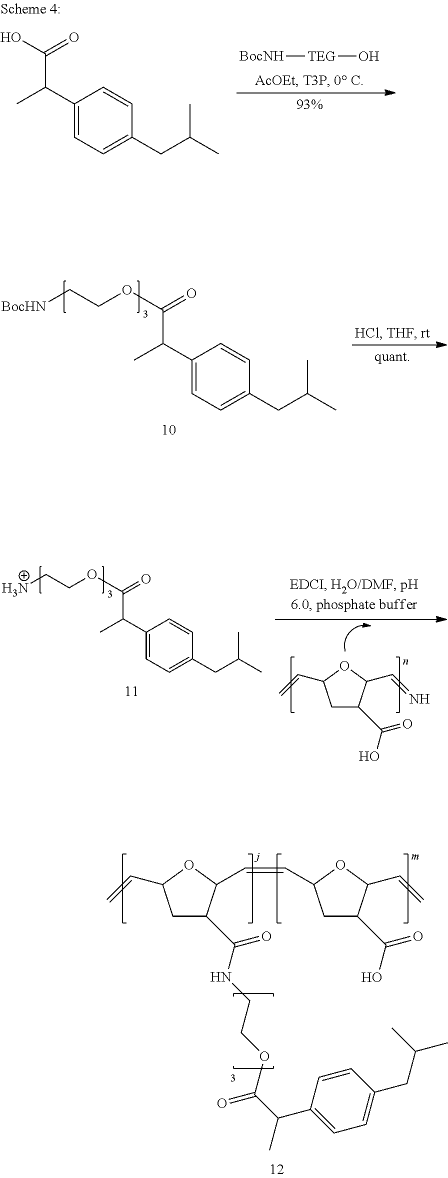

2-(2-(2-(tert-butoxycarbonyl)ethoxy)ethoxy)ethyl 2-(4-isobutylphenyl)propanoate (10). Ibuprofen (0.91 g, 4.44 mmol), tert-butyl 2-(2-(2-hydroxyethoxy)ethoxy)ethylcarbamate (TEG) (1 g, 4.0 mmol) were dissolved in ethyl acetate (15 mL) at 0° C. To the solution was added T3P (1 propane phosphonic acid anhydride) (1.4 g, 4.41 mmol), and reaction was stirred overnight, warming to rt. The reaction was subsequently filtered, and washed with NaHCO₃ solution. Organic layer was dried over Na₂SO₄, filtered, and the volatiles evaporated. The residue was purified by silica gel chromatography (DCM/MeOH—98/2). The product 10 was a white solid (93% yield, 1.63 g, 3.73 mmol). ¹H NMR (400 MHz, CDCl₃) δ=0.82 (d, 6H), 1.39 (s, 9H), 1.41 (d, 3H), 1.78 (septet, 1H), 2.38 (d, 2H), 3.21 (br s, 2H), 3.42 (br, 6H), 3.58 (br, 2H), 3.62 (q, 1H), 4.12-4.18 (m, 2H), 4.98 (br s, 1H), 7.15 (dd, 4H). ¹³C NMR (100 MHz, CDCl₃) δ=16.4, 22.7, 28.6, 29.1, 39.2, 40.2, 40.8, 64.9, 71.2, 71.3, 71.4, 79.7, 128.3, 129.2, 132.7, 156.1, 173.68. HRMS: m/z calcd for C₂₀H₃₁NO₄ [M+Na]⁺: 460.2675. found: 460.2683.

2-[2-(4-Isobutylphenyl)propanoyloxy]ethanaminium chloride (11). Compound 10 (1.0 g, 2.28 mmol) was dissolved in dry THF (10 mL) and dry HCl (3 mL, 2M in dioxane) was added to the solution at rt. The reaction was stirred overnight, filtered, and the filtrand washed with cold ether (3×10 mL), affording 11 as a white powder in quantitative yield (850 mg, 2.28 mmol). ¹H NMR (300 MHz, D₂O) δ=0.95 (d, 6H), 1.42 (d, 3H), 1.80 (septet, 1H), 2.41 (d, 2H), 3.18 (m, 2H), 3.40-3.80 (br, 9H), 4.22 (m, 2H), 7.04 (dd, 4H), 8.25 (br, 3H). ¹³C NMR (75 MHz, D₂O) δ=16.4, 22.9, 29.4, 40.4, 41.8, 45.5, 65.5, 69.8, 70.3, 75.0, 127.9, 129.3, 138.6, 141.0, 173.0. HRMS: m/z calcd for C₁₅H₂₃NO₂ [M+H]⁺: 338.2326. found: 338.23252.

Synthesis of Viscosupplement carrying ester-pro-Ibuprofen (12). Polyacid (100 mg, MW=2 MDa) was dissolved in phosphate buffer (10 mL, pH 6, 0.1M). DMF was added (2.5 mL), and the solution was cooled to 0° C. To the solution was added 11 (41 mg, 0.143 mmol) dissolved in H₂O (0.5 mL), followed by EDCI (30 mg, 0.17 mmol) dissolved in H₂O (0.5 mL). The reaction was stirred overnight allowing it to warm to rt. The solution was precipitated into ice-cold 1M HCl (150 mL) and filtered. The filtrand was redissolved in phosphate buffer (pH 7.4), and dialyzed for 96 h at 4° C. (membrane MW cut-off 300 kDa) in UltraPur water. The water was lyophilized off, affording the modified polymer as a white foam.

### Example 5

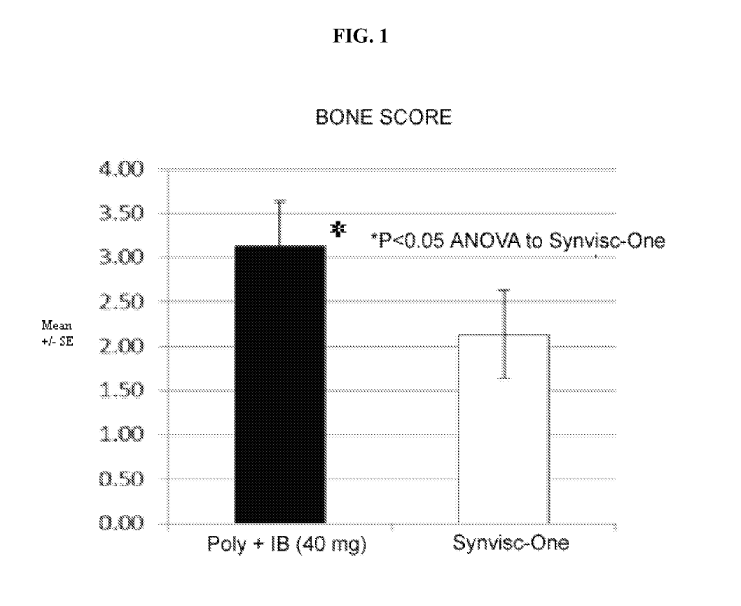

A polymer-ibuprofen conjugate was evaluated for its ability to alter the load bearing capacity of an arthritic joint in canines. FIG. 1 illustrates the results from a study that evaluated the efficacy of a polymer-ibuprofen conjugate in increasing the load bearing ability and bone score of joints, using a canine model of osteoarthritis. Briefly, dogs were treated with a polymer-ibupropen conjugate, at a concentration of 40 mg. Synvisc One®, a cross-linked hyaluronan supplment currently available via prescription, was used as a control in this study. As illustrated by FIG. 1, the polymer-ibuprofen conjugate (poly-IB) exhibited equal or higher bone score than Synvisc One®, when administered at a dose of 20 mg. Measurements of the percent width of the medial femur were used to calculate the bone score, which is a measure of bone density. For example, the following scoring was used: 0=normal; 1=up to 10% of femur width has thickened trabeculae; 2=11-30% of femur width has thickened trabeculae; 3=31-60% of femur width has thickened trabeculae; 4=61-90% of femur width has thickened trabeculae; 5=>91% of femur width has thickened trabeculae. Greater bone density indicates greater load bearing ability of joint and indicates a lesser degree of pain upon the application of weight (load). Conjugation of a non-steroidal anti-inflammatory drug (NSAID) such as ibuprofen to the polymers was observed to increase femur width which correlates to a higher bone score. Conjugation also provides a direct route for delivering a drug to the site of inflammation. Taken together these results suggest that administration of the conjugate may be an effective therapeutic regimen for treating osteoarthritis.

### EQUIVALENTS

The use of the terms “a” and “an” and “the” and similar referents in the context of describing the elements (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Additionally the phrase “consisting essentially of” will be understood to include those elements specifically recited and those additional elements that do not materially affect the basic and novel characteristics of the claimed invention. The phrase “consisting of” excludes any element not specifically specified.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A polymer comprising at least one monomeric unit represented by Formula I, II, or III: 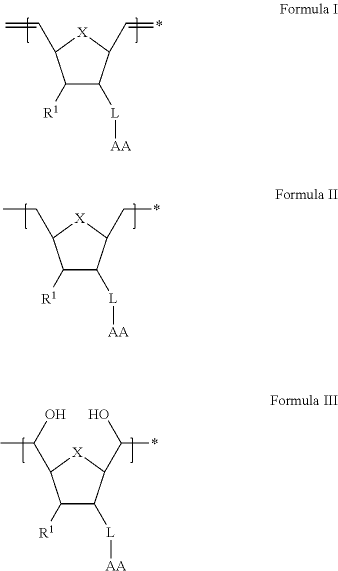
wherein:
X is CH₂ or O;
R¹ is H, COOR², COCH₃, CONHR², OR² or SR²;
L comprises a bond or a linker group of formula —C(O)—R⁶—, —C(O)NH—R⁶—NH—, —C(O)NH—R⁶—S—, —C(O)NH—R⁶—O—, —C(O)S—R⁶—NH—, —C(O)S—R⁶—S—, —C(O)S—R⁶—O—, —C(O)O—R⁶—NH—, —C(O)O—R⁶—S—, or —C(O)S—R⁶—O—;
AA is an active agent derived from a growth factor, a cytokine, a small molecule, an analgesic, an anesthetic, an antimicrobial agent, an antibacterial agent, an antiviral agent, an antifungal agent, an antibiotic, an anti-inflammatory agent, an antioxidant, an antiseptic agent, and any combination thereof;
each R² is independently a cation, protein, growth factor, enzyme, cytokine, antibody, single chain antibody, antibody fragment or (CH₂CH₂O)_{n′}R⁴;
each R³ is independently trityl, 4-methyltrityl or 2 pyridyl;
each R⁴ is independently CH₂NHR⁵, CH₂N(R⁵)₂, CH₂CH₂N(R⁵)₂, CH₂CO₂R⁵, or CH₂CH₂CO₂R⁵;
each R⁵ is independently a cation, protein, enzyme, growth factor, cytokine, antibody, single or a chain antibody;
each R⁶ is independently an alkylene, alkylene oxide, alkylene sulfide, poly(alkylene oxide), and
the active agent AA includes a carboxylic acid group.

2-7. (canceled)

8. The polymer of claim 1, wherein R² is an alkali metal, or an alkaline earth metal.

9. The polymer of claim 1, wherein R² is Li, Na, K, Cs, Ca, Mg, or Ba.

10. (canceled)

11. The polymer of claim 1, wherein AA is derived from ibuprofen, aspirin (acetylsalicylic acid), diflunisal, salsalate, naproxen, fenoprofen, ketoprofen, flurbiprofen, oxaprozin, indomethacin, sulindac, etodolac, ketorolac, diclofenac, mefenamic acid, meclofenamic acid, flufenamic acid, or tolfenamic acid.

12. A polymer comprising at least one monomeric unit represented by Formula IA, IIA, or IIIA: 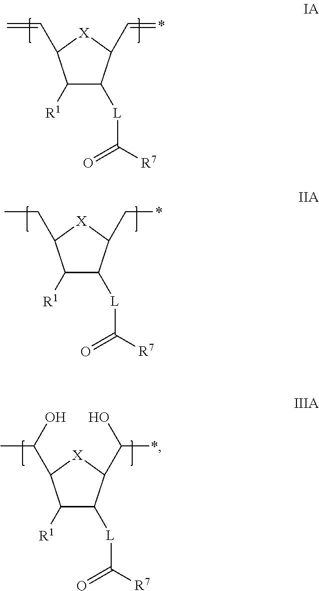
wherein:
L comprises a linker group of formula —C(O)—R⁶—, —C(O)OR⁶—, —C(O)NH—R⁶—, or —C(O)NH—R⁶—NH—;
R⁶ is an alkylene, alkylene oxide, alkylene sulfide, poly(alkylene oxide); and
R⁷ is a group of formula: 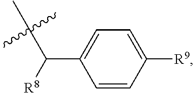
R⁸ is H or alkyl; and
R⁹ is alkyl an alkylene group.

13. (canceled)

14. The polymer of claim 12, wherein R⁸ is methyl, ethyl, n-propyl, or iso-propyl; and R⁹ is methyl, ethyl, n-propyl, iso-propyl, n-butyl, iso-butyl, sec-butyl, tert-butyl, pentyl, neo-pentyl, iso-pentyl, or hexyl.

15. (canceled)

16. The polymer of claim 1 that is a polymer of Formula IB: 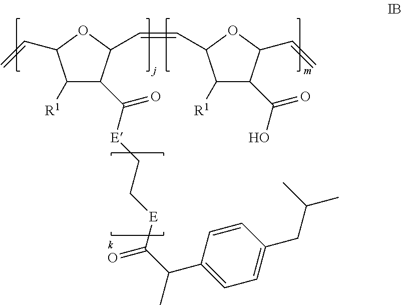
wherein:
R¹ is H, C(O)OH, C(O)OR², or OR².
E is S, O, or NH;
E′ is S, O, or NH;
m is from 100 to 200,000;
j is from 100 to 200,000; and
k is from 1 to 500.

17. (canceled)

18. The polymer of claim 16, wherein E′ is NH.

19. The polymer of claim 16, wherein R¹ is H, E is S or NH and k is 1.

20. (canceled)

21. The polymer of claim 16, wherein R¹ is H, E is O and k is 1-3.

22. The polymer of claim 1 having a molecular weight from about 2.3×10⁶ g/mol to about 2.9×10⁶ g/mol.

23-24. (canceled)

25. The polymer of claim 1, wherein from about 1% to about 99% of the monomeric units comprise an AA group.

26-27. (canceled)

28. A pharmaceutical composition comprising an effective amount of at least one polymer of claim 1 and at least one pharmaceutically acceptable carrier.

29. A method comprising: administering an effective amount of a polymer of claim 1 to a subject in need thereof.

30-37. (canceled)

38. The method of claim 29, wherein the administering comprises performing local administration of an effective amount of the polymer to a tissue of the subject, the tissue is a diseased or injured synovial joint, and the polymer is used as a viscosupplement.

39. A process comprising:
contacting a first polymer comprising at least one monomeric unit of Formula V, VI, or VII with a compound of Formula VIII to produce a second polymer having at least one monomeric unit of Formula IC, IIC, or IIIC;
wherein: 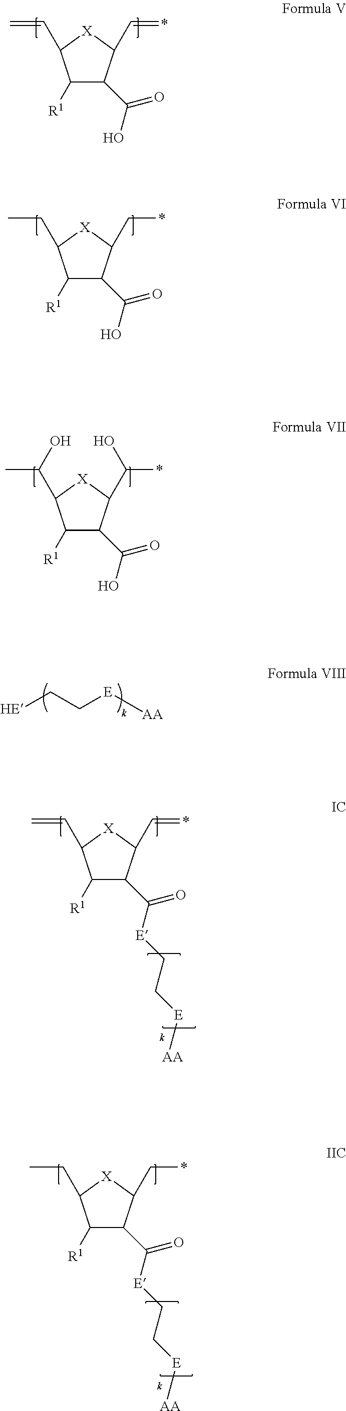 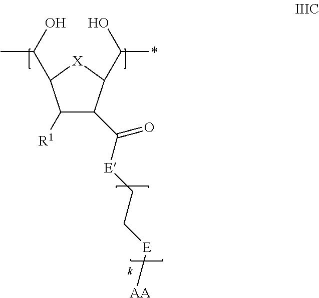
X is CH₂ or O;
R¹ is H, COOR², COCH₃, CONHR², OR² or SR²;
E is O, S, or NR¹⁰;
E′ is O, S, NR¹⁰, or ⁺NHR¹⁰X′, wherein X′ is an anion;
k is an integer from 1 to 500;
AA is a group derived from an active agent;
each R² is independently H, a cation, an alkyl, an alkenyl, an alkynyl, COCH₃, CH₂CH₂OH, CH₂CH₂OR³, an amino acid, a small peptide, a large peptide, enzyme, protein, growth factor, cytokine, antibody, single chain antibody, antibody fragment, COCCH₃═CH₂, COCH═CH₂, CH₂CO₂H, CH₂CH₂CO₂H, CH₂CH₂SH, CH₂CH₂SR³ or (CH₂CH₂O)_{n′}R³;
n′ is an integer from 1 to 2000;
each R³ is independently trityl, 4-methyltrityl or 2 pyridyl;
each R⁴ is independently H, an alkyl, an alkenyl, an alkynyl, COCCH₃═CH₂, COCH═CH₂, CH₂CHO, CH₂CH₂CHO, CO₂H, CO₂R⁵, CH₂CO₂H, CH₂CH₂CO₂H, CH₂NH₂, CH₂NHR⁵, CH₂N(R⁵)₂, CH₂CH₂NH₂, CH₂CH₂NHR, CH₂CH₂N(R⁵)₂, SH, CH₂CO₂R⁵, or CH₂CH₂CO₂R⁵;
each R⁵ is independently maleimide, an amino acid, a small peptide, a large peptide, protein, enzyme, growth factor, cytokine, antibody, single chain antibody, phosphate, sulfate, choline, or an activated ester; and
each R¹⁰ is independently H or alkyl.

40. The process of claim 39, wherein the first polymer is a compound of Formula V and the second polymer is a compound of Formula IC.

41-43. (canceled)

44. The process of claim 39, wherein AA is a group of formula: 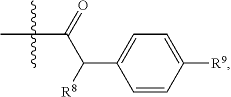
wherein R⁸ is H or alkyl; and R⁹ is alkyl.

45-47. (canceled)

48. The process of claim 39, wherein the second polymer is represented as Formula ID, IID, or IIID: 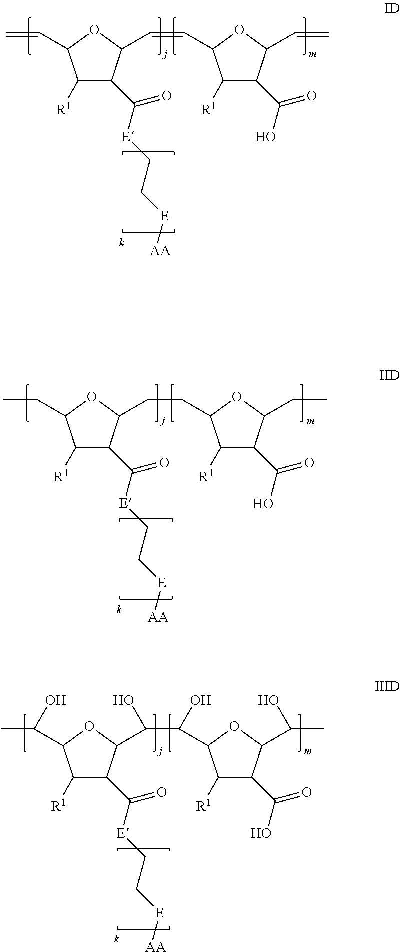
wherein: m is from 100 to 200,000 and j is from 100 to 200,000.

49. The process of claim 48, wherein from about 1% to about 25% of the monomeric units in Formula ID, IID, or IIID comprise the AA group.

50-58. (canceled)
